(19) United States
(12) Patent Application Publication
(10) Pub. No.: US 2021/0146064 A1
(43) Pub. Date: May 20, 2021

(54) SYRINGE

(71) Applicant: KRUI Medical AB, Helsingborg (SE)

(72) Inventor: Per KNUTSSON, Helsingborg (SE)

(73) Assignee: KRUI Medical AB, Helsingborg (SE)

(21) Appl. No.: 17/048,260

(22) PCT Filed: Apr. 15, 2019
(86) PCT No.: PCT/EP2019/059592

§ 371 Date: Oct. 16, 2020

(30) Foreign Application Priority Data
Apr. 18, 2018 (SE) 1850449-8

(51) Int. Cl. A61M 5/50

(52) U.S. Cl. CPC A61M 5/5066

(57) ABSTRACT

A syringe (100) comprising a barrel (111) with a lumen is provided. The barrel (111) is adapted to contain a drug, the barrel (111) having a distal end wall portion (198) and a tubular wall (112) extending proximally from said distal end wall portion (198), whereby the barrel (111) extends along an axis (1000). The distal end wall portion (198) has an opening (199) extending into a syringe head (140) adapted to receive a syringe needle, said syringe head (140) having a lumen smaller in transversal cross-section than the barrel lumen; the syringe (100) further comprising: a plunger (196) extending along the axis, said plunger shaft being displaceable along the axis (1000) within said barrel (111).

### TECHNICAL FIELD

The present invention relates to a syringe comprising a plunger for preventing re-usage of the syringe which can be manufactured at a cost similar to a conventional syringe.

### BACKGROUND

Misusage of syringes is a common issue, of the 16 billion injections administered each year over 2 million patients and users are infected with severe blood transmittable diseases, such as HIV, Hepatitis B and C. In order to prevent the emergence of disease and infection due to misusage of syringes some syringes are equipped with arrangements for preventing re-usage. This may be especially important with regards to prevent re-usage by addicts. In some instances the prevention is achieved via an arrangement, wherein the needle is refracted into the syringe barrel after one usage whereby additional injections with the syringe are made impossible. In some instances the prevention is achieved by arrangements which destroy the syringe needle after one usage. There are also instances where the prevention is achieved via introduced weaknesses in the syringe plunger which may cause the plunger to deform or brake and thereby preventing re-usage. In further instances re-usage may be prevented by a piece of metal molded into the plunger.

One advantage with such arrangements is that the risk for injuries and disease due to contact with the syringe needle in for example the disposal process is more or less eliminated. However, the arrangements enabling the retracting or destruction of the needles are often complex and more costly to produce. Since the cost for syringes often is a driving factor for the re-usage of conventional syringes, there is a need for more cost-efficient solutions for providing the prevention of re-usage. Another issue with current arrangements for preventing re-usage of syringes is that the volume of the drug in the syringe is not utilized properly. The arrangements are usually disposed inside the cylinder barrel of the syringe, are space-consuming and further limit the volume of the contained drug which is possible to inject through the needle.

Examples of prior art attempts are e.g. US20060064060 A1, which is easy to manipulate into re-using and has an easily manipulated distance before locking; US20050038394 A1, which has an inefficient distal end, a short, easily manipulated distance before locking and a very weak plunger; and US20050240149 A1, which functions unreliably depending on the applied force and has an easily manipulated distance before locking. All of these applications are abandoned, possibly due to weaknesses only realized during manufacturing.

Hence, there is a need to provide a syringe which addresses the disadvantages and shortcomings of the prior art in general and to provide a non-reusable syringe which can be manufactured as well as used in a cost-efficient manner.

### SUMMARY OF THE INVENTION

Accordingly, the present invention preferably seeks to mitigate, alleviate or eliminate one or more of the above-identified deficiencies in the art and disadvantages singly or in any combination and solves at least the above mentioned problems by proposing a solution according to the appended independent claims. Advantageous embodiments are defined in the appended dependent claims.

In a first of its aspects, this disclosure therefore presents a syringe comprising a barrel with a lumen. The barrel is adapted to contain a drug and has a distal end wall portion and a tubular wall extending proximally from the distal end wall portion, whereby the barrel extends along an axis. The distal end wall portion has an opening extending into a syringe head adapted to receive a syringe needle. The syringe head has a lumen smaller in transversal cross-section than the barrel lumen. The syringe further comprises a plunger extending along the axis, its plunger shaft being displaceable along the axis within the barrel. The plunger comprises a plunger top. The plunger top is adapted to be in fluid-tight engagement with the tubular wall of the barrel. The plunger top comprises a protrusion, whereby the protrusion is adapted to fit in the lumen of the syringe head. The protrusion comprises an elongated recess transversally through the protrusion extending along the axis and a retaining portion formed by a portion of the protrusion wider in transversal cross-section than the remainder of the protrusion. The protrusion is adapted to be retained inside the lumen of the syringe head upon insertion by means of an interaction surface. The syringe head comprises a retaining wall having a distally facing interaction surface and the plunger is adapted to be prevented from being pulled in a direction away from the distal end wall portion by means of a proximally facing interaction surface of the retaining portion locking against the distally facing interaction surface of the retaining wall. The retaining wall is formed by a wall section inside the syringe head. The wall section forms a section of the lumen of the syringe head smaller in the transversal cross-section than the remainder of the lumen of the syringe head. The distally facing interaction surface of the retaining wall is formed by a distally facing interaction surface of the wall section. The interaction surfaces are arranged to engage with each other when the plunger top is at a distance D2 from the distal end wall portion.

One advantage of this is that it prevents re-use of the syringe by users avoiding activating the lock by making the syringe inefficient and dangerous to use in that way.

In some embodiments, the distance D2 is between 0.1-7.5 mm.

Alternatively, the distance D2 may range from 1-7.5 mm or 2-7.5 mm and preferably be approximately 7.5 mm.

In some embodiments, the interaction surfaces are arranged to engage with each other when the distal end of the protrusion is at a distance D1 of between 0.1-7.5 mm from the distal end of the syringe head.

One advantage of this is that by placing the locking mechanism so that the retaining portion is entirely within the lumen of the syringe head, it is harder to tamper with by sticking elongated objects into the lumen of the syringe head and pushing the retaining portion back in a proximal direction past the retaining wall thereby enabling additional injections. Additionally, 7.5 mm is the ISO standard length of Luer syringe heads, so any excess distance would be wasted. Alternatively, the distance D1 may range from 1-7.5 mm or 2-7.5 mm and preferably be approximately 7.5 mm.

In some embodiments, the distances D1, D2 are equal.

One advantage of this is that the distances D1, D2 reach zero at the same time in order to efficiently deposit all of the medicament in the barrel and the syringe head. Close to the entire volume of the barrel and the syringe head may thus be emptied of the drug, leading to less spillage and a syringe which is more cost-efficient to use.

In a second of its aspects, this disclosure presents a syringe comprising a barrel with a lumen. The barrel is adapted to contain a drug and has a distal end wall portion and a tubular wall extending proximally from the distal end wall portion, whereby the barrel extends along an axis. The distal end wall portion has an opening extending into a syringe head adapted to receive a syringe needle. The syringe head has a lumen smaller in transversal cross-section than the barrel lumen. The syringe further comprises a plunger extending along the axis, its plunger shaft being displaceable along the axis within the barrel. The plunger comprises a plunger top. The plunger top is adapted to be in fluid-tight engagement with the tubular wall of the barrel. The plunger top comprises a protrusion, whereby the protrusion is adapted to fit in the lumen of the syringe head. The protrusion comprises an elongated recess extending along the axis and a retaining portion formed by a portion of the protrusion wider in transversal cross-section than the remainder of the protrusion. The protrusion is adapted to be retained inside the lumen of the syringe head upon insertion by means of an interaction surface. The syringe head comprises a retaining wall having a distally facing interaction surface and the plunger is adapted to be prevented from being pulled in a direction away from the distal end wall portion by means of a proximally facing interaction surface of the retaining portion locking against the distally facing interaction surface of the retaining wall. The retaining wall is formed by a wall section inside the syringe head. The wall section forms a section of the lumen of the syringe head smaller in the transversal cross-section than the remainder of the lumen of the syringe head. The distally facing interaction surface of the retaining wall is formed by a distally facing interaction surface of the wall section. At least one of the interaction surfaces has a surface roughness arithmetic average (Ra) between 1.1-9 μm.

One advantage of this is that by increasing the surface roughness of at least one of the interaction surfaces, their frictional force will increase which leads to a greater retaining effect for the interaction surfaces.

In some embodiments, the increased surface roughness of said at least one of the interaction surfaces is achieved using sparking, etching or blasting.

One advantage of this is that specific options may be cheaper or achieve a better result.

In a third of its aspects, this disclosure presents a syringe comprising a barrel with a lumen. The barrel is adapted to contain a drug and has a distal end wall portion and a tubular wall extending proximally from the distal end wall portion, whereby the barrel extends along an axis. The distal end wall portion has an opening extending into a syringe head adapted to receive a syringe needle. The syringe head has a lumen smaller in transversal cross-section than the barrel lumen. The syringe further comprises a plunger extending along the axis, its plunger shaft being displaceable along the axis within the barrel. The plunger comprises a plunger top. The plunger top is adapted to be in fluid-tight engagement with the tubular wall of the barrel. The plunger top comprises a protrusion, whereby the protrusion is adapted to fit in the lumen of the syringe head. The protrusion comprises an elongated recess extending along the axis and a retaining portion formed by a portion of the protrusion wider in transversal cross-section than the remainder of the protrusion. The protrusion is adapted to be retained inside the lumen of the syringe head upon insertion by means of an interaction surface. The syringe head comprises a retaining wall having a distally facing interaction surface and the plunger is adapted to be prevented from being pulled in a direction away from the distal end wall portion by means of a proximally facing interaction surface of the retaining portion locking against the distally facing interaction surface of the retaining wall. The retaining wall is formed by a wall section inside the syringe head. The wall section forms a section of the lumen of the syringe head smaller in the transversal cross-section than the remainder of the lumen of the syringe head. The distally facing interaction surface of the retaining wall is formed by a distally facing interaction surface of the wall section. The elongated recess of the protrusion has rounded edges.

One advantage of this is that it prevents air bubbles from sticking to the walls of the elongated recess. It may also reduce the wall thickness of the protrusion, which makes it faster and cheaper to manufacture using e.g. injection molding.

In some embodiments, the rounded edges create divots in the radius of the top of the retaining portion.

One advantage of this is that it makes it easier for the retaining portion to penetrate the retaining wall.

In a fourth of its aspects, this disclosure presents a syringe comprising a barrel with a lumen. The barrel is adapted to contain a drug and has a distal end wall portion and a tubular wall extending proximally from the distal end wall portion, whereby the barrel extends along an axis. The distal end wall portion has an opening extending into a syringe head adapted to receive a syringe needle. The syringe head has a lumen smaller in transversal cross-section than the barrel lumen. The syringe further comprises a plunger extending along the axis, its plunger shaft being displaceable along the axis within the barrel. The plunger comprises a plunger top. The plunger top is adapted to be in fluid-tight engagement with the tubular wall of the barrel. The plunger top comprises a protrusion, whereby the protrusion is adapted to fit in the lumen of the syringe head. The protrusion comprises an elongated recess extending along the axis and a retaining portion formed by a portion of the protrusion wider in transversal cross-section than the remainder of the protrusion. The protrusion is adapted to be retained inside the lumen of the syringe head upon insertion by means of an interaction surface. The syringe head comprises a retaining wall having a distally facing interaction surface and the plunger is adapted to be prevented from being pulled in a direction away from the distal end wall portion by means of a proximally facing interaction surface of the retaining portion locking against the distally facing interaction surface of the retaining wall. The retaining wall is formed by a wall section inside the syringe head. The wall section forms a section of the lumen of the syringe head smaller in the transversal cross-section than the remainder of the lumen of the syringe head. The distally facing interaction surface of the retaining wall is formed by a distally facing interaction surface of the wall section. The plunger comprises at least two kerfs adapted to deform or break when the plunger is pulled in a direction away from the distal end wall portion after retaining of the protrusion of the plunger top inside the lumen of the syringe head so as to disallow displacement of the plunger top by means of movement of the plunger.

One advantage of this is that it makes the plunger more stable during manufacturing, transportation and use. In some embodiments, the at least two kerfs are arranged symmetrically around the center of the plunger.

One advantage of this is that it makes the plunger even more stable during manufacturing, transportation and use.

In some embodiments, the at least two kerfs are arranged near at least two different edges of a cross-section of the plunger.

One advantage of this is that it makes the plunger even more stable during manufacturing, transportation and use.

In a fifth of its aspects, this disclosure presents a syringe comprising a barrel with a lumen. The barrel is adapted to contain a drug and has a distal end wall portion and a tubular wall extending proximally from the distal end wall portion, whereby the barrel extends along an axis. The distal end wall portion has an opening extending into a syringe head adapted to receive a syringe needle. The syringe head has a lumen smaller in transversal cross-section than the barrel lumen. The syringe further comprises a plunger extending along the axis, its plunger shaft being displaceable along the axis within the barrel. The plunger comprises a plunger top. The plunger top is adapted to be in fluid-tight engagement with the tubular wall of the barrel. The plunger top comprises a protrusion, whereby the protrusion is adapted to fit in the lumen of the syringe head. The protrusion comprises an elongated recess extending along the axis and a retaining portion formed by a portion of the protrusion wider in transversal cross-section than the remainder of the protrusion. The protrusion is adapted to be retained inside the lumen of the syringe head upon insertion by means of an interaction surface. The syringe head comprises a retaining wall having a distally facing interaction surface and the plunger is adapted to be prevented from being pulled in a direction away from the distal end wall portion by means of a proximally facing interaction surface of the retaining portion locking against the distally facing interaction surface of the retaining wall. The retaining wall is formed by a wall section inside the syringe head. The wall section forms a section of the lumen of the syringe head smaller in the transversal cross-section than the remainder of the lumen of the syringe head. The distally facing interaction surface of the retaining wall is formed by a distally facing interaction surface of the wall section. Each proximally facing interaction surface of the retaining portion of the protrusion is one straight plane that has different angles toward the longitudinal axis of the protrusion depending on the rotation around the longitudinal axis.

One advantage of this is that, the protrusion may be injection molded while simultaneously being easy to eject from the injection mold cavity.

In some embodiments, the different angles toward the longitudinal axis of the protrusion vary between 70° and 90°.

One advantage of this is that the retaining effect is more resilient to deformation.

In a sixth of its aspects, this disclosure presents a syringe comprising a barrel with a lumen. The barrel is adapted to contain a drug and has a distal end wall portion and a tubular wall extending proximally from the distal end wall portion, whereby the barrel extends along an axis. The distal end wall portion has an opening extending into a syringe head adapted to receive a syringe needle. The syringe head has a lumen smaller in transversal cross-section than the barrel lumen. The syringe further comprises a plunger extending along the axis, its plunger shaft being displaceable along the axis within the barrel. The plunger comprises a plunger top. The plunger top is adapted to be in fluid-tight engagement with the tubular wall of the barrel. The plunger top comprises a protrusion, whereby the protrusion is adapted to fit in the lumen of the syringe head. The protrusion comprises an elongated recess extending along the axis and a retaining portion formed by a portion of the protrusion wider in transversal cross-section than the remainder of the protrusion. The protrusion is adapted to be retained inside the lumen of the syringe head upon insertion by means of an interaction surface. The syringe head comprises a retaining wall having a distally facing interaction surface and the plunger is adapted to be prevented from being pulled in a direction away from the distal end wall portion by means of a proximally facing interaction surface of the retaining portion locking against the distally facing interaction surface of the retaining wall. The retaining wall is formed by a wall section inside the syringe head. The wall section forms a section of the lumen of the syringe head smaller in the transversal cross-section than the remainder of the lumen of the syringe head. The distally facing interaction surface of the retaining wall is formed by a distally facing interaction surface of the wall section. The plunger top comprises an O-ring adapted to be in fluid-tight engagement with the tubular wall.

One advantage of this is that it is easier to manufacture.

In some embodiments, the O-ring is made of silicon with a hardness of 50 to 100 IRHD.

One advantage of this is that it ensures the purity of the medicament for a long time and greatly reduces the need for silicon lubrication.

In some embodiments, the O-ring is press fit radially 0.1 to 0.2 mm, preferably 0.15 mm.

One advantage of this is that a more consistent seal is achieved and lower friction will occur during movement.

In some embodiments, the O-ring is disposed circumferentially around a radial guiding protrusion of the plunger top, whereby the radial guiding protrusion is adapted to protrude into an inner envelope surface of the O-ring so as to deform a portion of said inner envelope surface radially outwards.

One advantage of this is that it keeps the O-ring in place and prevents it from rolling due to friction between it and the tubular wall of the barrel when the plunger is displaced along the axis.

In some embodiments, the O-ring has a substantially planar distal surface.

One advantage of this is that this simplifies manufacturing, specifically the attachment of the plunger top.

In some embodiments, the syringe is the first aspect in combination with the second aspect and/or in combination with the third aspect and/or in combination with the fourth aspect and/or in combination with the fifth aspect and/or in combination with the sixth aspect.

One advantage is that all of the advantages of these aspects may be combined.

In some embodiments, after the interaction surfaces have engaged with each other, the distal end of the protrusion is displaceable along the axis between the position of interaction surface engagement and the distal end of the syringe head.

One advantage of this is that it is easier to manufacture.

In some embodiments, the plunger comprises at least one kerf adapted to deform or break when the plunger is pulled in a direction away from the distal end wall portion after retaining of the protrusion of the plunger top inside the lumen of the syringe head so as to disallow displacement of the plunger top by means of movement of the plunger.

One advantage of this is that it further limits the re-usability of the syringe.

In some embodiments, the kerf is arranged along the axis at a distance D3 away from the plunger top, whereby the distance D3 is longer than 1 mm and shorter than the distance D4 between the proximal end of the barrel and a stopping portion of the tubular wall.

One advantage of this is that the minimum distance yields a plunger that is sturdier and needs less care to be taken during manufacturing. The maximum distance is because otherwise the kerf will be located on a part of the plunger which extends beyond the barrel of the syringe when the plunger is fully withdrawn. This is disadvantageous because it exposes the kerf to outside forces without the protection of the tubular wall of the barrel.

In some embodiments, the plunger is made of a rigid material adapted to break at the kerf when the plunger is pulled in a direction away from the distal end wall portion.

One advantage of this is that after the kerf breaks, the syringe may be disposed of in a more efficient manner.

In some embodiments, the plunger is made of Polypropylene with a relatively high E-modulus.

One advantage of this is that in combination with a protrusion which has an elongated recess extending through its distal tip, an increased stiffness of the plunger and accordingly also the protrusion enhances the retaining effect of the retaining portion.

In some embodiments, the plunger is made of an elastic material adapted to viscoelastically deform at the kerf when the plunger is pulled in a direction away from the distal end wall portion.

One advantage of this is that less force may be needed to render the syringe non-reusable.

In some embodiments, the plunger is made of Polypropylene with a relatively low E-modulus.

One advantage of this is that in combination with a closed-off recess, the material is more inclined to deform so as to easily allow for the retaining portion to pass through the opening as well as the retaining wall when the protrusion is pushed through the syringe head.

In some embodiments, the distally facing interaction surface of the retaining wall and/or the proximally facing interaction surface of the retaining portion extend in an inclined direction extending proximally from the axis.

One advantage of this is that the retaining effect of the interaction surfaces is increased.

In some embodiments, the proximally facing interaction surface of the retaining portion of the protrusion and the distally facing interaction surface of the retaining wall are disposed so as to allow for proximal displacement of the plunger before the retaining contact between the proximally facing interaction surface of the retaining portion and the distally facing interaction surface of the retaining wall occur.

One advantage of this is that this is easier to manufacture.

In some embodiments, the distal end wall portion of the barrel comprises reinforcing ribs.

One advantage of this is that it increases sturdiness and saves on materials. It may also reduce the wall thickness of the distal end wall portion, which makes it faster and cheaper to manufacture using e.g. injection molding.

In some embodiments, the elongated recess is substantially conical and wider in transversal cross-section towards the distal tip of the protrusion.

One advantage of this is that the robustness of the retaining functionality is increased and a stiffer material may be used due to the shape of the recess effectively directing the deformation so as to achieve a force directed radially outward as well as proximally. The direction of the force further serves to increase the stability and robustness of the retaining portion.

In some embodiments, the elongated recess is closed-off along the axis.

One advantage of this is increasing the stability of the plunger during the compression and retaining since all of the surrounding walls provide the retaining and compression effect necessary to retain the protrusion after insertion in the syringe head as well as during insertion of the protrusion into the syringe head. Furthermore, compared to not having a closed-off recess the production process is less susceptible to quality issues since the design is not as reliant on exact tolerances.

In some embodiments, the elongated recess extends through a distal tip of the protrusion.

One advantage of this is that it is easier to manufacture.

In some embodiments, each proximally facing interaction surface of the retaining portion of the protrusion is divided into a first and second interaction surface that have different angles toward the longitudinal axis of the protrusion.

One advantage of this is that the protrusion may be injection molded while simultaneously being easy to eject from the injection mold cavity.

In some embodiments, the first and second interaction surface have different angles toward the longitudinal axis of the protrusion depending on the rotation angle around the longitudinal axis.

One advantage of this is that it is much harder to reduce the retaining effect of the interaction surfaces.

In some embodiments, each proximally facing interaction surface of the retaining portion of the protrusion is divided into a first and second interaction surface that have different distances to a distal tip of the protrusion.

One advantage of this is that, it is much harder to reduce the retaining effect of the interaction surfaces.

In some embodiments, each proximally facing interaction surface of the retaining portion of the protrusion is curved.

One advantage of this is that it is much harder to reduce the retaining effect of the interaction surfaces.

In some embodiments, the plunger comprises at least one ridge arranged to support a cavity in the plunger.

One advantage of a ridge is that it prevents an unintended break of the plunger e.g. during manufacturing.Further advantages will be apparent from the detailed description as well as the appended dependent claims.

### BRIEF DESCRIPTION OF THE DRAWINGS

These and other aspects, features and advantages of which the invention is capable, will be apparent and elucidated from the following description of embodiments of the present invention, reference being made to the accompanying drawings, in which

FIG. 1 is a longitudinal section view of a syringe according to one example;

FIG. 2 is a longitudinal section view of the syringe according to one example, where a plunger is fully withdrawn from a barrel of the syringe;

FIG. 3 is a longitudinal section view of the syringe according to one example, where a protrusion is penetrating the lumen of a syringe head;

FIG. 4a is a longitudinal section view of the syringe according to one example, where a proximally facing interaction surface of a retaining portion is locking against a distally facing interaction surface of a retaining wall;

FIG. 4b is a longitudinal section view of the locking interaction surfaces according to one example, where the distally facing interaction surface of the retaining wall has an inclined orientation;

FIG. 4c is a longitudinal section view of the locking interaction surfaces according to one example, where the distally facing interaction surface of the retaining wall does not have an inclined orientation;

FIG. 5 is a longitudinal section view of the syringe according to one example, where the plunger is fully inserted into the barrel of the syringe;

FIG. 6a is a longitudinal section view of the syringe according to one example, where a kerf in the plunger is starting to weaken the plunger;

FIG. 6b is a longitudinal section view of the syringe according to one example, where the plunger has broken at the kerf;

FIG. 7 is a longitudinal view of the syringe according to one example, where different distances are defined;

FIG. 8 is an isometric side view of the barrel according to one example, where a distal end wall portion of the barrel comprises reinforcing ribs;

FIG. 9 is an isometric proximal view of the distally facing interaction surface of the retaining wall according to one example;

FIG. 10a is an isometric side view of the protrusion according to one example, where an elongated recess of the protrusion has rounded edges;

FIG. 10b is an isometric distal view of the protrusion according to one example, where the elongated recess of the protrusion has rounded edges;

FIG. 10c is a longitudinal section view of the protrusion according to one example, where the elongated recess of the protrusion has rounded edges;

FIG. 10d is a longitudinal section view of the protrusion according to one example, where the elongated recess of the protrusion has rounded edges;

FIG. 11a is an isometric side view of the protrusion according to one example, where the elongated recess of the protrusion does not have rounded edges;

FIG. 11b is an isometric distal view of the protrusion according to one example, where the elongated recess of the protrusion does not have rounded edges;

FIG. 11c is a longitudinal section view of the protrusion according to one example, where the elongated recess of the protrusion does not have rounded edges;

FIG. 11d is a longitudinal section view of the protrusion according to one example, where the elongated recess of the protrusion does not have rounded edges;

FIG. 12 is a distal view of the proximally facing interaction surface of the retaining portion according to one example, where it has a surface roughness arithmetic average (Ra) of 3.5 μm;

FIG. 13a are side views of the protrusion according to one example, where different proximally facing interaction surfaces have different angles towards the protrusion;

FIG. 13b is a longitudinal section view along A-A of the protrusion of FIG. 13a;

FIG. 14 are longitudinal section views and an isometric distal view of the protrusion according to one example, where the proximally facing interaction surface is curved;

FIG. 15a are different views of the protrusion according to one example where the elongated recess of the protrusion does not have rounded edges, with a cross-section view along A-A of the elongated recess and a longitudinal section view along B-B of the distal end of the protrusion;

FIG. 15b are different views of the protrusion according to one example where the elongated recess of the protrusion has rounded edges, with a cross-section view along A-A of the elongated recess and a longitudinal section view along B-B of the distal end of the protrusion;

FIG. 16a shows schematic illustrations of an example syringe during different stages of use, where the plunger of the syringe has two kerfs in a cavity of the plunger and ridges arranged to support the cavity;

FIG. 16b are different views of the syringe of FIG. 16a;

FIG. 17a are side cross section views of an example syringe during different stages of use, where the plunger of the syringe has two kerfs in a cavity of the plunger and ridges arranged to support the cavity;

FIG. 17b are different views of the syringe of FIG. 17a;

FIG. 18a are side cross section views of an example syringe during different stages of use, where the plunger of the syringe has three kerfs;

FIG. 18b are different views of the syringe of FIG. 18a;

FIG. 19a is a side view and a longitudinal section view of the syringe with an O-ring that is press fit radially at a high radius; and

FIG. 19b is a side view and a longitudinal section view of the syringe with an O-ring that is press fit radially at a low radius.

### DETAILED DESCRIPTION OF THE EMBODIMENTS

Embodiments of the present invention will be described in more detail below with reference to the accompanying drawings in order for those skilled in the art to be able to carry out the invention. The invention may, however, be embodied in many different forms and should not be construed as limited to the embodiments set forth herein. Rather, these embodiments are provided so that this disclosure will be thorough and complete, and will fully convey the scope of the invention to those skilled in the art. The embodiments do not limit the invention, but the invention is only limited by the appended patent claims. Furthermore, the terminology used in the detailed description of the particular embodiments illustrated in the accompanying drawings is not intended to be limiting of the invention.

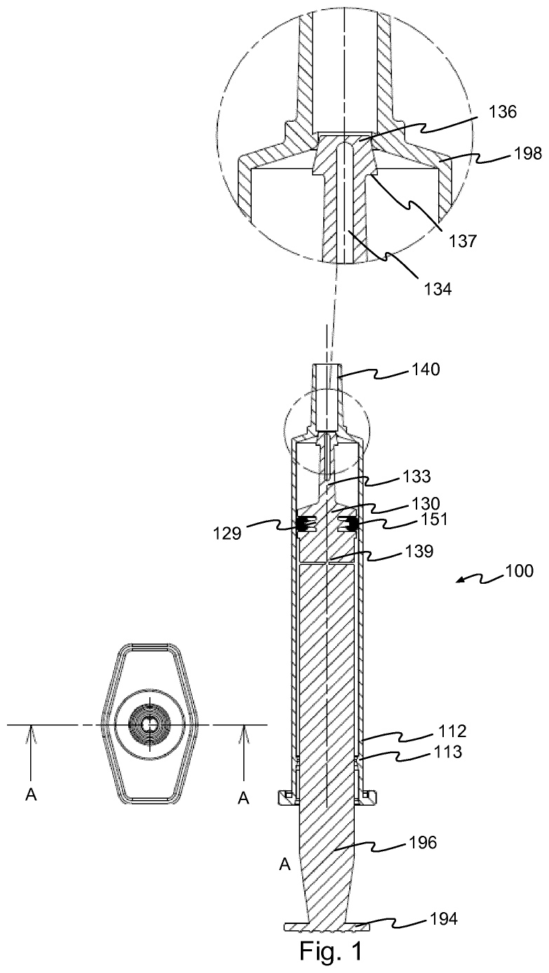

The FIGS. 1-6 show a cross-section along A-A as seen in FIG. 1. Referring to FIG. 1, a longitudinal section view of a syringe 100 according to an example is shown. The syringe 100 comprises a barrel 111 with a lumen, preferably of a cylindrical shape, which is adapted to contain a drug in liquid form to be injected into a patient. The barrel 111 has a distal end wall portion 198 and a tubular wall 112 extending proximally from the distal end wall portion 198.

The syringe 100 extends along an axis 1000, which defines the direction in which the barrel 111 extends, i.e. the barrel 111 extends along the axis 1000. The syringe 100 has a syringe head 140 i.e. a syringe hub for receiving a syringe needle intended for injecting the drug into a patient. The syringe head 140 may be a Luer, such as an exterior Luer, for example a Luer slip or a Luer lock. The syringe head 140 further has a lumen which preferably is smaller in transversal cross-section than the barrel lumen. Fluid communication to the syringe needle is enabled by the distal end wall portion 198 having an opening 199 extending into the syringe head 140 i.e. the lumen of the syringe head 140.

Injection of the drug is achieved by moving of a plunger 196. The plunger 196 extends along the axis 1000 and extends into a thumb rest 194. Said plunger 196 is displaceable along the axis 1000 within the barrel 111.

The plunger 196 comprises a plunger top 130, the plunger top 130 being adapted to be in fluid-tight engagement with the tubular wall 112 of the barrel 111. The tubular wall 112 comprises a stopping portion 113 a distance D4 away from its proximal end which is a protrusion extending into the lumen of the barrel 111 adapted to stop the plunger top 130 from moving proximally beyond the stopping portion 113. The plunger top 130 comprises a protrusion 133, whereby the protrusion 133 is adapted to fit in the lumen of the syringe head 140.

The plunger top 130 may comprise an O-ring 151 adapted to be in fluid-tight engagement with the tubular wall 112. Due to the implementation of the O-ring 151 the remainder of the plunger 196 may be manufactured in one piece and simply be equipped with the O-ring 151 to guarantee the sealing contact with the tubular wall 112. Thereby, the plunger 196 does not have to be manufactured by assembly of several components, resulting in a more cost-efficient manufacturing process.

Preferably, the O-ring 151 has a substantially planar distal surface, which increases the contact surface between the O-ring 151 and the plunger top 130. The increased contact surface serves to prevent rolling of the sealing ring 151 during displacement of the plunger 196 for example during injection.

This effect may be further accentuated by the O-ring 151 being disposed circumferentially around a radial guiding protrusion 129 of the plunger top 130, whereby the radial guiding protrusion 129 is adapted to protrude into an inner envelope surface of the O-ring 151 so as to deform a portion of said inner envelope surface radially outwards. The O-ring 151 is thus retained and prevented from rolling due to the friction between it and the tubular wall 112 of the barrel 111 when the plunger 196 is displaced along the axis 1000.

The radial guiding protrusion 129 may be disposed on the plunger top 130, whereby the radial guiding protrusion 129 is formed by two radial recesses disposed distally and proximally of said protrusion 129. The radial guiding protrusion 129 then extends into the inner envelope surface of the O-ring 151 so as to retain the O-ring 151. In other words, the guiding protrusion 129 is adapted to deform the inner envelope surface of the O-ring 151 so as to allow the O-ring 151 to partially encase the radial protrusion 129.

Advantageously, the protrusion 133 comprises an elongated recess 134 transversally through the protrusion 133 extending along the axis 1000 and a retaining portion 136 formed by a portion of the protrusion wider in transversal cross-section than the remainder of the protrusion 133. The elongated recess 134 is preferably a closed elongated recess 134, such that the distal end of the protrusion 133 encloses the elongated recess 134.

To further enhance retaining effect achieved by the protrusion 133, the protrusion 133 may be substantially conical and thinner in transversal cross-section towards the distal tip of the protrusion 133. Thereby, the robustness of the retaining functionality is increased and a stiffer material may be used due to the shape of the recess effectively directing the deformation so as to achieve a force directed radially outward as well as proximally. The direction of the force further serves to increase the stability and robustness of the retaining portion 136.

To prevent re-use of the syringe, the protrusion 133 is adapted to be retained inside the lumen of the syringe head 140 upon insertion by means of the retaining portion 136. The syringe head 140 further comprises a retaining wall 148 having a distally facing interaction surface 147 and the plunger 196 is adapted to be prevented from being pulled in a direction away from the distal end wall portion 198 by means of a proximally facing interaction surface 137 of the retaining portion 136 locking against said distally facing interaction surface 147 of the retaining wall 148. When the protrusion 133 is moved in a distal direction through the syringe head 140 it is thus compressed axially by means of the elongated recess 134 so as to pass through the portion of the syringe head 140 comprising the retaining wall 148. However, when the plunger is pulled backwards, i.e. in a proximal direction, the retaining portion 136 abuts to the retaining wall 148 and prevents further proximal movement. The abutting between the interaction surfaces 137, 147 of said retaining portion 136 and retaining wall 148 prevents re-usage since the plunger 196 cannot be used to suck in additional drugs after one usage.

Thereby, a less complex and more robust solution for preventing re-usage of a syringe 100 may be achieved due to the user not being able to pull the protrusion 133 out of the syringe head 140.

FIG. 1 shows a position of the plunger 196 where the protrusion 133 abuts the opening 199 of the distal end wall portion 198 i.e. the lumen of the syringe head 140. By pushing the plunger 196 further distally along the axis 1000, the protrusion 133 will penetrate the opening 199 of the distal end wall portion 198 and activate the lock of the interaction surfaces 137, 147.

The retaining portion 136 is preferably constituted by a portion of the protrusion 133 being wider in transversal cross-section situated at the distal tip of protrusion 133. The proximally facing interaction surface 137 is thus formed by the proximal surface of the portion of the retaining portion 136 which protrudes radially from the remainder of the protrusion 133.

With advantage, the syringe head 140 may further comprise at least one axial passage in fluid communication with the barrel 111, the axial passage being located laterally of the protrusion 133 when the protrusion 133 is positioned in the lumen of the syringe head 140. Preferably, the axial passage is formed by a recess in a wall of the syringe head 140.

Thereby a syringe 100 which enables usage of close to the entire volume of the drug contained in the barrel 111 is provided due to the drug being able to enter through the syringe head 140 even when the protrusion 133 is inserted therein by flowing through said at least one axial passage.

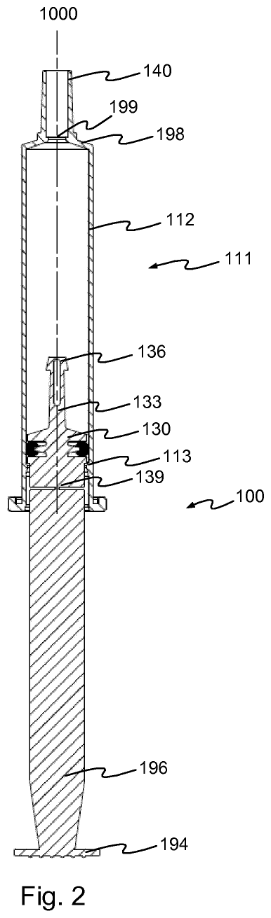

FIG. 2 shows a longitudinal section view of the syringe 100 according to one example, where the plunger 196 is fully withdrawn from the barrel 111 of the syringe 100. At this point, the plunger top 130 abuts the stopping portion 113 of the tubular wall 112. The plunger 196 extends into a thumb rest 194. Preferably, the plunger 196 has a stiffening rib extending along its length to increase the robustness of the syringe 100. The plunger 196 comprises a plunger top 130, which further comprises the protrusion 133.

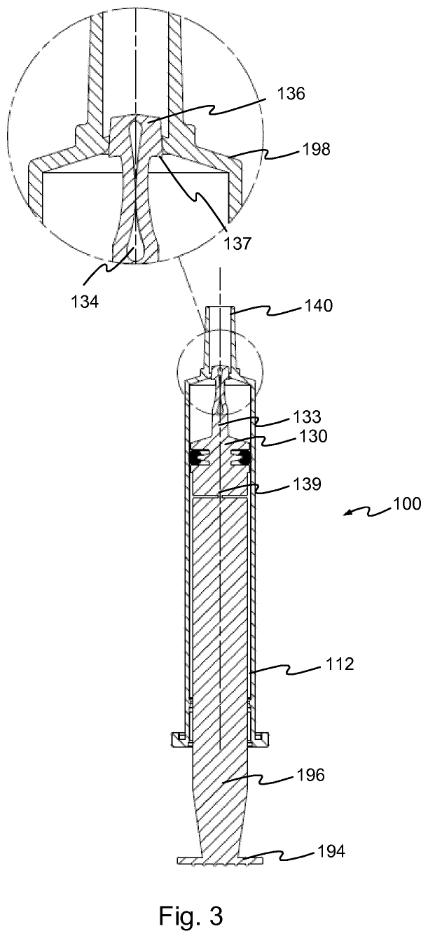

FIG. 3 shows a longitudinal section view of the syringe 100 according to one example, where the protrusion 133 is penetrating the lumen of the syringe head 140. When the plunger top 130 with the protrusion 133 is moved in a distal direction through the syringe head 140 it is thus compressed axially and consequentially radially by means of the recess 134, so as to pass through the portion of the syringe head 140 comprising the retaining wall 148. FIG. 3 depicts the recess 134 in a compressed state during insertion of the protrusion 133 through the portion of the syringe head 140 comprising the retaining wall 148 in a distal direction.

With advantage, the retaining portion 136 may have a substantially tapered shape, whereby the distal end of said retaining portion 136 may be substantially smaller in transversal cross-section, i.e. a radial direction orthogonal to the axis 1000, than the proximal end of said retaining portion 136. This makes it easier for the retaining portion 136 to pass through the section of the syringe head 140 with the retaining wall 148 when the plunger 196 is moved in a distal direction during injection, making the syringe 100 more user-friendly. This is achieved through the tapered shape being easier to be radially compressed when the plunger 196 is moved in the direction of the tapering.

Again referring to FIG. 3, an example of the syringe 100 with a retaining portion 136 which utilizes a closed-off elongated recess 134 is shown. Hence, the elongated recess 134 is closed-off along the axis 1000, said recess 134 may thus be an elongated through-recess extending through the protrusion 133 and along the axis 1000 being closed-off in every other direction. This is particularly advantageous due to the closed-off design increasing the stability of the plunger 196 during the compression and retaining since all of the surrounding walls provide the retaining and compression effect necessary to retain the protrusion 133 after insertion in the syringe head 140 as well as during insertion of the protrusion 133 into the syringe head 140. Furthermore, compared to not having a closed-off recess 134 the production process is less susceptible to quality issues since the design is not as reliant on exact tolerances.

Alternatively, the elongated recess 134 may be closed-off along the axis 1000 and every other direction except one, leading to a through-hole in a radial direction which is elongated along the axis 1000.

Advantageously, the thickness of the material surrounding the recess 134 along the axis 1000 is very thin. Preferably, the thickness of said material may be between 0.5-1 mm, even more preferably between 0.6-0.8 mm and most preferably between 0.6-0.7 mm. This allows for a very low friction force to enable the locking of the protrusion 133, since the walls are easy to deform, i.e. compress, so as to allow the retaining portion 136 to pass the retaining wall 148 of the syringe head 140.

Alternatively, the elongated recess 134 may extend through a distal tip of the protrusion 133. Thus, the resilience of the protrusion 133 allows for the split end to deform inwardly when the protrusion 133 is moved distally through the syringe head 140 during injection. The proximal movement may thus be prevented by the retaining portion 136 abutting a retaining wall 148 which may be disposed inside the syringe head 140. Thereby, a simple and cost-efficient syringe 100 which does not require any complex manufacturing methods or assembly while still providing a non-reusability functionality can be achieved.

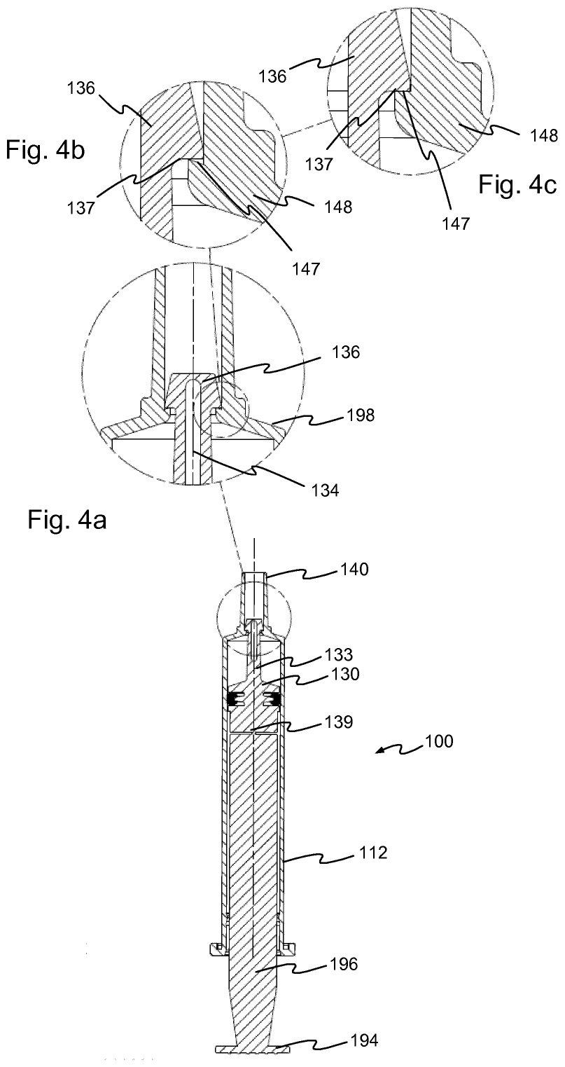

FIG. 4a shows a longitudinal section view of the syringe 100 according to one example, where the proximally facing interaction surface 137 of the retaining portion 136 is locking against the distally facing interaction surface 147 of the retaining wall 148. When the plunger 196 is pulled backwards, i.e. in a proximal direction, the retaining portion 136 abuts the retaining wall 148 and prevents further proximal movement. The abutting between the interaction surfaces 137, 147 of said retaining portion 136 and retaining wall 148 prevents re-usage since the plunger 196 cannot be used to suck in additional drugs after one usage.

Thereby, a less complex and robust solution for preventing re-usage of a syringe 100 can be achieved due to the user not being able to pull the protrusion 133 out of the syringe head 140.

With further reference to FIGS. 4a-c, the retaining wall 148 may be formed by a wall section inside the syringe head 140, said wall section 148 forming a section of the lumen of the syringe head 140 smaller in the transversal cross-section than the remainder of the lumen of the syringe head 140, whereby the distally facing interaction surface 147 of the retaining wall is formed by a distally facing interaction surface of the wall section 148.

The proximally facing interaction surface 137 of the retaining portion 136 of the protrusion 133 and the distally facing interaction surface 147 of the retaining wall 148 are disposed so as to allow for proximal displacement of the plunger 196 before the retaining contact between said proximally facing interaction surface 137 of the retaining portion 136 and the distally facing interaction surface 147 of the retaining wall 148 occur. The distance D1 which the plunger 196 is allowed to move proximally before the interaction surfaces 137, 147 abut; after the retaining portion 136 of the protrusion 133 has passed the retaining wall 148 when the plunger 196 has been moved in a distal direction first; serves to disallow access to the plunger 196 by means of for example sticking elongated objects and pushing the retaining portion 136 back in a proximal direction past the retaining wall 148 thereby enabling additional injections.

Further referring to FIG. 4b, the distally facing interaction surface 147 of the retaining wall 148 extends in an inclined direction extending proximally from the axis 1000. Either or both of the interaction surfaces 137, 147 may extend in an inclined direction extending proximally from the axis 1000. Due to the tilted orientation of the interaction surfaces 137, 147 and direction extending proximally from the axis 1000, the edges of said interaction surfaces 137, 147 will interlock when subjected to an axial tensile torque exceeding a certain level. If the interaction surfaces 137, 147 extend substantially orthogonal to the axis 1000 there is a risk for the edges to glide away from each other, whereby no interlocking takes place. Hence, the inclined interaction surfaces 137, 147 serve to achieve a more robust and efficient non-reusability functionality.

Furthermore, said inclined orientation of the proximally facing interaction surface 137 serves to enable radial inward deformation of the edges of said proximally facing interaction surface 137 when the protrusion 133 is moved distally through the syringe head 140. This reduces the resistance for the injection movement of the plunger 196 compared to having a conventional cylindrical plunger 196. Hence, the aforementioned inclined direction of the proximally facing interaction surface 137 enables a more user-friendly syringe.

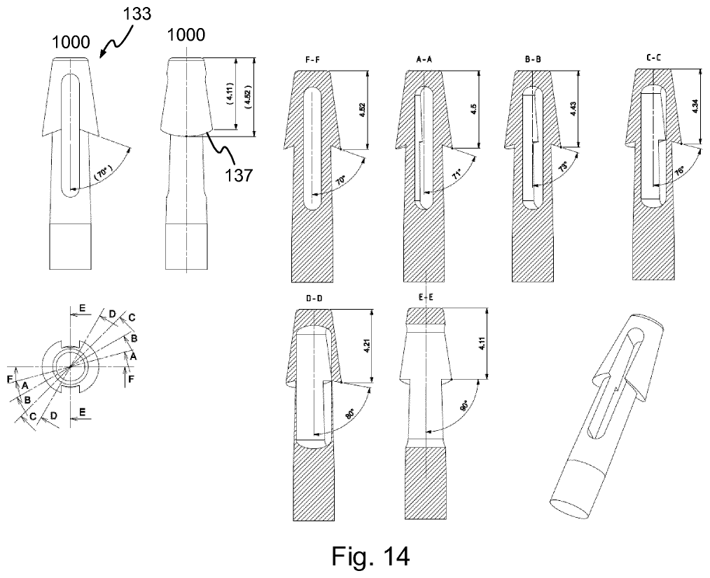

However, the proximally facing interaction surface 137 extends orthogonally to the protrusion 133. This means that with an inclined orientation of the proximally facing interaction surface 137 an undercut is formed. During manufacturing, it is common for the undercut to be damaged, deformed or broken. It is not cost efficient enough to manufacture these components in a way that will not damage the undercut, therefore interaction surfaces 137, 147 without an inclined orientation is preferred. These however have historically had problems with sliding apart during use. This will be discussed further in regards to FIG. 14.

FIG. 4c shows a longitudinal section view of the locking interaction surfaces 137, 147 according to one example, where the interaction surfaces 137, 147 do not have an inclined orientation. This increases the surface interface area of the interaction surfaces 137, 147.

This may be beneficial in an embodiment where one or both of the interaction surfaces 137, 147 have an increased surface roughness. This is used to prevent the interaction surfaces 137, 147 from sliding apart from each other when the plunger 196 is pulled backwards, i.e. in a proximal direction. The increased surface roughness may have a surface roughness arithmetic average (Ra) between 1.1-9 μm. The Ra must be above 1.1 μm as otherwise the surface/s is/are not rough enough to prevent sliding. The Ra must be below 9 μm as the interaction surfaces 137, 147 are not large enough to accommodate a larger Ra.

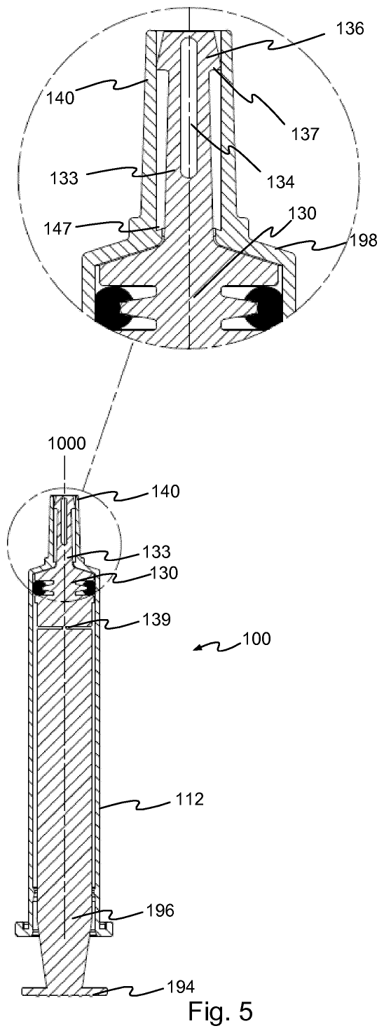

FIG. 5 shows a longitudinal section view of the syringe 100 according to one example, where the plunger 196 is fully inserted into the barrel 111 of the syringe 100. The distal end of the protrusion 133 is aligned with the top of the syringe head 140. Any medicament that was once contained in the barrel 111 of the syringe 100 should have been injected into a patient and the syringe 100 should be empty.

At this point, the syringe 100 is used and should be prevented from being re-used. The plunger 196 may be pulled backwards, i.e. in a proximal direction, a distance D1 corresponding to the distance between the interaction surfaces 137, 147 when the plunger 196 is fully inserted into the barrel 111 of the syringe 100, as shown in FIG. 5.

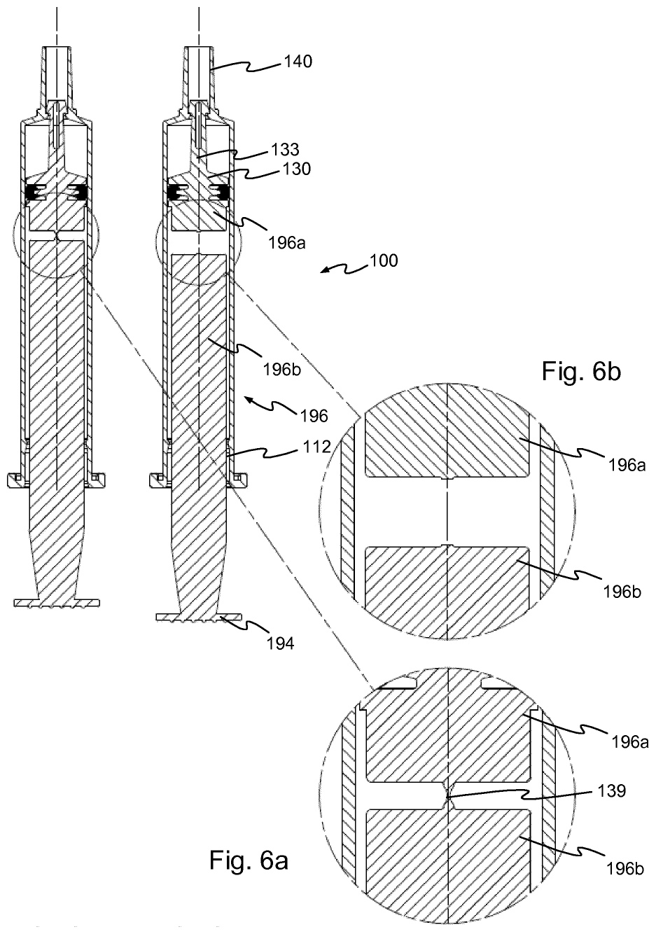

Turning to FIGS. 6a-b, the plunger 196 may comprise a kerf 139 arranged a proximal distance D3 along the axis 1000 from the plunger top 130. The kerf 139 may be adapted to deform or break when the plunger 196 is pulled in a direction away, i.e. proximally away, from the distal end wall portion 198 after retaining the protrusion 133 of the plunger top 130 inside the lumen of the syringe head 140 so as to disallow displacement of the plunger top 130 by means of moving the plunger 196.

The kerf 139 thus connects a distal portion 196a and a proximal portion 196b of the plunger 196, whereby the kerf 139 is a thin section of the plunger 196 extending between the distal 196a and proximal 196b portions of the plunger 196. Accordingly, the breaking or deformation at the kerf 139 serves to disallow the movement of the distal portion 196a of the plunger 196 which comprises the protrusion 133 by means of moving the proximal portion 196b of the plunger 196 which is movable by means of pulling the thumb-rest 194 of the plunger 196.

With further reference to FIGS. 4a-c as well as FIGS. 6a-b, an example of a syringe 100 in which the retaining portion 136 extends beyond the retaining wall 148 of the syringe head 140 is shown. The interaction surfaces 137, 147 are locking against each other, preventing the plunger 196 and more specifically the distal portion 196a of the plunger 196 from being displaced further proximally. The proximal movement may thus be prevented by the retaining portion 136 abutting to a retaining wall 148 which may be disposed inside the syringe head 140. If the plunger 196 is further proximally pulled beyond this limit, the situation in FIG. 6a may happen. If the plunger 196 is pulled even further, the situation in FIG. 6b may happen. Alternatively, the situation in FIG. 6b may happen directly.

With reference to FIGS. 6a, a longitudinal section view of the syringe 100 according to one example is shown, whereby the kerf 139 is starting to weaken the plunger 196. The plunger 196 may be made of an elastic material adapted to viscoelastically deform at the kerf 139 when the plunger 196 is pulled in a direction away from the distal end wall portion 198. The viscoelastic deformation is thus unable to transfer load through the plunger top 130 whereby it becomes impossible for the user to pull the protrusion 133 out of the syringe head 140 by moving the plunger 196 proximally since the material at the kerf 139 will simply continue to extend and elongate due to the abutting contact between the interaction surfaces 137, 147.

The plunger 196 may be made of Polypropylene with a relatively low E-modulus. This is particularly advantageous in combination with a closed-off recess 134 since the material is more inclined to deform so as to easily allow for the retaining portion 136 to pass through the opening 199 as well as the retaining wall 148 when the protrusion 133 is pushed through the syringe head 140. Thus, a more user-friendly syringe is achieved.

If the plunger 196 continues to be pulled, the kerf 139 will break resulting in the situation of FIG. 6b. FIG. 6b is a longitudinal section view of the syringe 100 according to one example, where the plunger 196 has broken at the kerf 139. The plunger 196 is completely separated into the distal 196a and proximal 196b portions. The distal portion 196a is limitedly displaceable along the axis 1000, limited by the locking action of the interaction surfaces 137, 147 and the distal end wall portion 198.

The proximal portion 196b is fully displaceable along the axis 1000, limited only in the distal direction by the distal portion 196a. The proximal portion 196b does not comprise the plunger top 130, which means that this portion 196b is not prevented from moving proximally beyond the stopping portion 113 of the tubular wall 112. The proximal portion 196b may therefore be completely removed from the barrel 111 of the syringe 100 and disposed of separately from the rest of the syringe 100. This conserves space in waste bins adapted for hazardous materials, which usually receive used syringes. The proximal portion 196b of the plunger 196 may be arranged to never be in contact with the medicament and may therefore be disposed of in a normal recycling bin.

Alternatively, the breaking of FIG. 6b may occur without the deformation of FIG. 6a as a precursor. In one embodiment, the plunger 196 is in a rigid material adapted to break at the kerf 139 when the plunger 196 is pulled in a direction away from the distal end wall portion 198. Thus, the material at the kerf 139 simply disconnects the distal portion 196a (which comprises the protrusion 133) and the proximal portion 196b, whereby it becomes impossible for the user to pull the protrusion 133 out of the syringe head 140 by moving the plunger 139.

The plunger 196 may be made of Polypropylene with a relatively high E-modulus. This is particularly advantageous in combination with a protrusion 133 that has an elongated recess 134 extending through its distal tip since an increased stiffness of the plunger 196 and accordingly also the protrusion 133 enhances the retaining effect of the retaining portion 136.

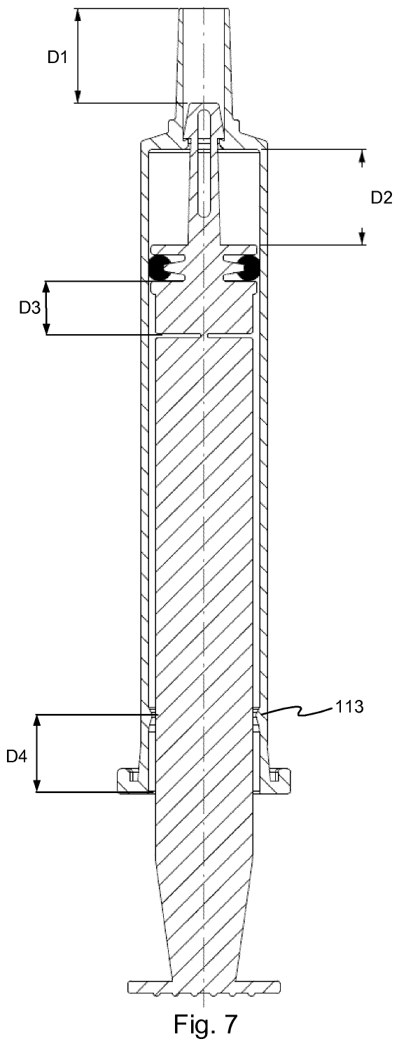

FIG. 7 shows a longitudinal view of the syringe 100 according to one example, where different distances are defined. The distance D1 is defined when the plunger 196 is in a position where the interaction surfaces 137, 147 are in a locking action. At this point, the distance D1 is the distance between the distal end of the protrusion 133 and the distal end of the syringe head 140. This distance D1 is determined by the placement along the axis 1000 of the retaining wall 148 within the lumen of the syringe top 140 as well as the size of the retaining portion 136 of the protrusion 133. The distance D1 defines how much the protrusion 133 is displaceable after the locking of the interaction surfaces 137, 147.

The distance D1 is preferably between 0.1-7.5 mm. The distance D1 is preferably not longer than 7.5 mm because 7.5 mm is the ISO standard length of Luer syringe heads, so any excess distance would be wasted. Alternatively, the distance D1 may range from 1-7.5 mm or 2-7.5 mm and preferably be approximately 7.5 mm.

A distance D2 is also defined when the plunger 196 is in a position where the interaction surfaces 137, 147 are in a locking action. At this point, the distance D2 is the distance between the plunger top 130 and the distal end wall portion 198 of the barrel 111 of the syringe 100. This distance D2 is determined by the length of the protrusion 133 and the placement along the axis 1000 of the retaining wall 148 within the lumen of the syringe top 140. The distance D2 defines how much medicament is left in the barrel 111 of the syringe 100 when the interaction surfaces 137, 147 lock and prevent proximal displacement of the plunger top 130.

The distance D2 is preferably between 0.1-7.5 mm. The distance D2 is preferably longer than 0.1 mm because otherwise close to the entire volume of the syringe 100 may be emptied without activating the lock. This is disadvantageous because users may then avoid activating the lock and be able to re-use the syringe 100 while still being able to use most of the volume of the syringe 100. The innovative feature of the distance D2 being at least 0.1 mm prevents re-use of the syringe 100 by users avoiding activating the lock by making the syringe 100 inefficient and dangerous to use in that way.

Alternatively, the distance D2 may range from 1-7.5 mm or 2-7.5 mm and preferably be approximately 7.5 mm. The distance D2 is also preferably equal to or approximately equal to the distance D1 between the distal end of the protrusion 133 and the distal end of the syringe head 140. This is because in normal use, the distances D1, D2 preferably reach zero at the same time in order to efficiently deposit all of the medicament in the barrel 111 and the syringe head 140. Close to the entire volume of the barrel 111 and the syringe head 140 may thus be emptied of the drug, leading to less spillage and a syringe which is more cost-efficient to use.

The distance D3 is the distance between the kerf 139 and the plunger top 130. This distance D3 is determined by the placement along the axis 1000 of the kerf 139. The kerf 139 may be placed freely along the entire length of the plunger 196. It is however preferable if the distance D3 is longer than 1 mm and shorter than the distance D4 between the proximal end of the barrel 111 and the stopping portion 113 of the tubular wall 112.

The distance D3 is preferably longer than 1 mm. This is because by shifting the kerf 139 away from the plunger top 130, manufacturing is made simpler, faster and sturdier. The kerf 139 is by design the weakest portion of the plunger 196. During manufacturing the plunger 196 receives the plunger top 130. This attachment may be made using a less delicate process if the kerf 139 is at least 1 mm away from the attachment point, i.e. the position of the plunger top 130. This also means that the kerf 139 may be made to more easily deform if it is under less stress during manufacturing, which makes the kerf 139 function better as intended.

The distance D3 is preferably shorter than the distance D4 between the proximal end of the barrel 111 and the stopping portion 113 of the tubular wall 112. This is because otherwise the kerf 139 will be located on a part of the plunger 196 that extends beyond the barrel 111 of the syringe 100 when the plunger 196 is fully withdrawn. This is disadvantageous because it exposes the kerf 139 to outside forces without the protection of the tubular wall 112 of the barrel 111.

The distance D4 is the distance between the proximal end of the barrel 111 and the stopping portion 113 of the tubular wall 112. This distance D4 is determined by the placement along the axis 1000 of the stopping portion 113. The stopping portion 113 may be placed freely along the entire length of the tubular wall 112. The distance D4 defines how far the plunger 196 may be pulled back and therefore also the maximum volume of the medicament in the barrel 111. It is preferable to have a short distance D4 because then more medicament may fit. However, it is also preferably to have a long distance D4 to allow for a longer D3, which increases the durability of the plunger 196.

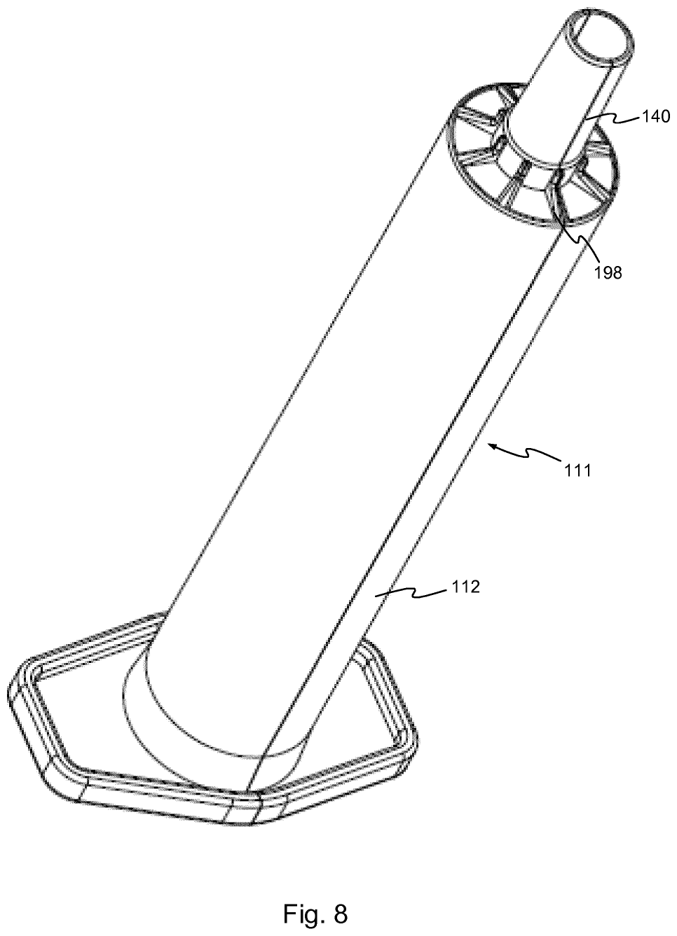

FIG. 8 shows an isometric side view of the barrel 111 according to one example, where the distal end wall portion 198 of the barrel 111 comprises reinforcing ribs. The reinforcing ribs are preferably made from a rigid polymer and extend between the tubular wall 112 of the barrel 111 and the syringe head 140. These reinforcing ribs may be added as additional support to the distal end wall portion 198 of the barrel 111. They may alternatively replace the distal end wall portion 198 of the barrel 111. This embodiment saves on material and may be easier to manufacture. This embodiment also reduces the wall thickness of the distal end wall portion 198, which makes it faster and cheaper to manufacture using e.g. injection molding.

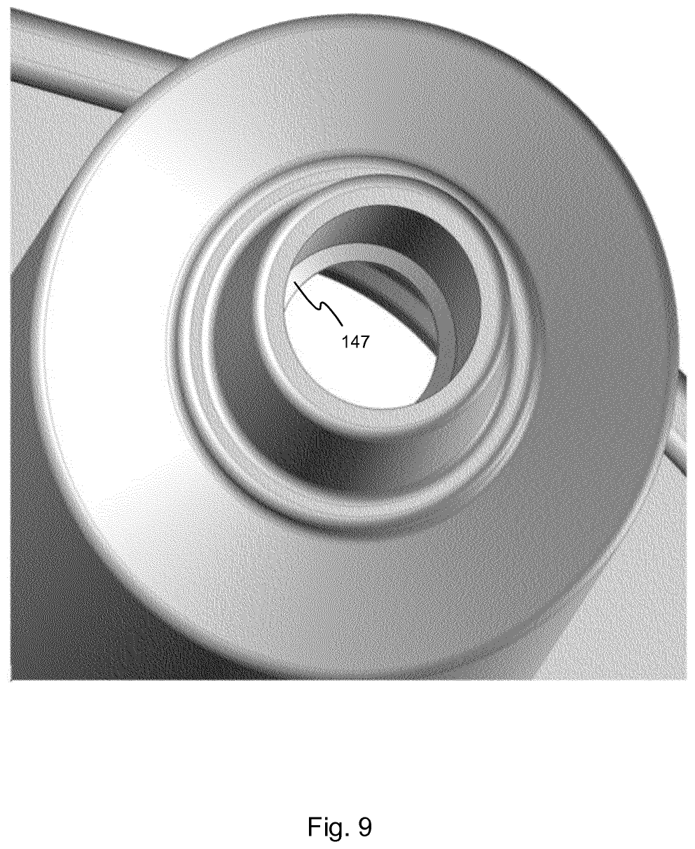

FIG. 9 shows an isometric proximal view of the distally facing interaction surface 147 of the retaining wall 148 according to one example. The interaction surface 147 extends symmetrically in 360 degrees. This symmetry allows the proximally facing interaction surface 137 of the retaining portion 136, which may be divided into two parts by the elongated recess 134 and therefore not rotationally symmetric, to be fully engaged by the distally facing interaction surface 147 regardless of the rotation of the plunger 196.

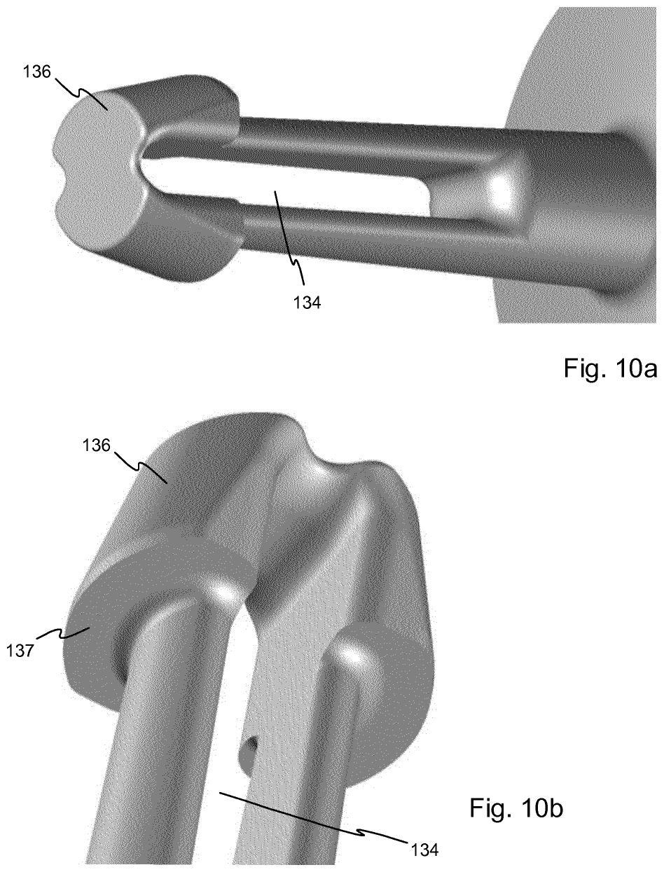

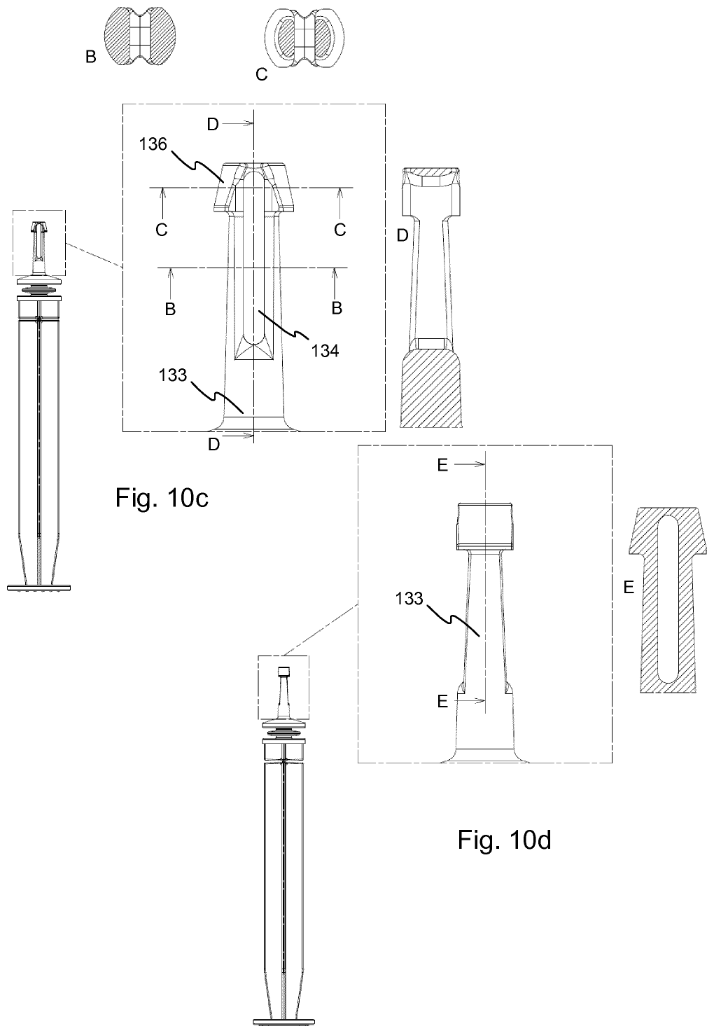

The FIGS. 10c-d and 11c-d show cross-sections along B-B, C-C, D-D and E-E as marked. FIGS. 10a-d show views of the protrusion 133 according to one example, where the elongated recess 134 of the protrusion 133 has rounded edges in a cross-section transversal to the longitudinal axis of the protrusion 133. The elongated recess 134 may also have a rounded distal and proximal end, in a longitudinal section of the protrusion 133. The elongated recess 134 is intended to be in contact with the medicament. Air bubbles may exist among the medicament and these may be dangerous to inject into a patient. The rounded edges of the elongated recess 134 are designed to guide air bubbles away from the elongated recess 134 so that air bubbles do not get stuck there.

The rounded edges may also impact the top of the retaining portion 136, creating divots in its radius as seen in FIGS. 10a-c. This may allow the retaining portion 136 to be more easily deformed. This makes it easier to penetrate the retaining wall 148. The rounded edges may also reduce the wall thickness of the protrusion 133, which makes it faster and cheaper to manufacture using e.g. injection molding.

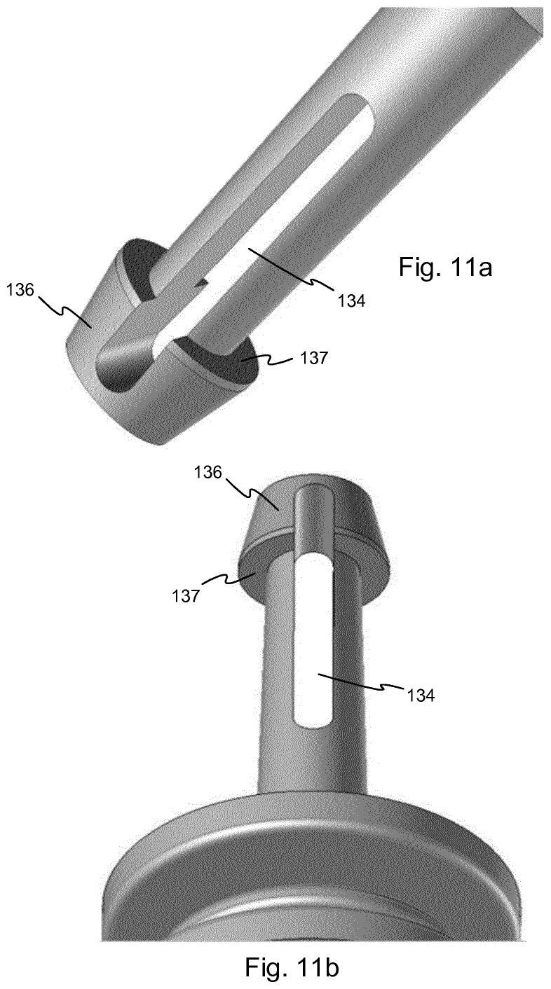

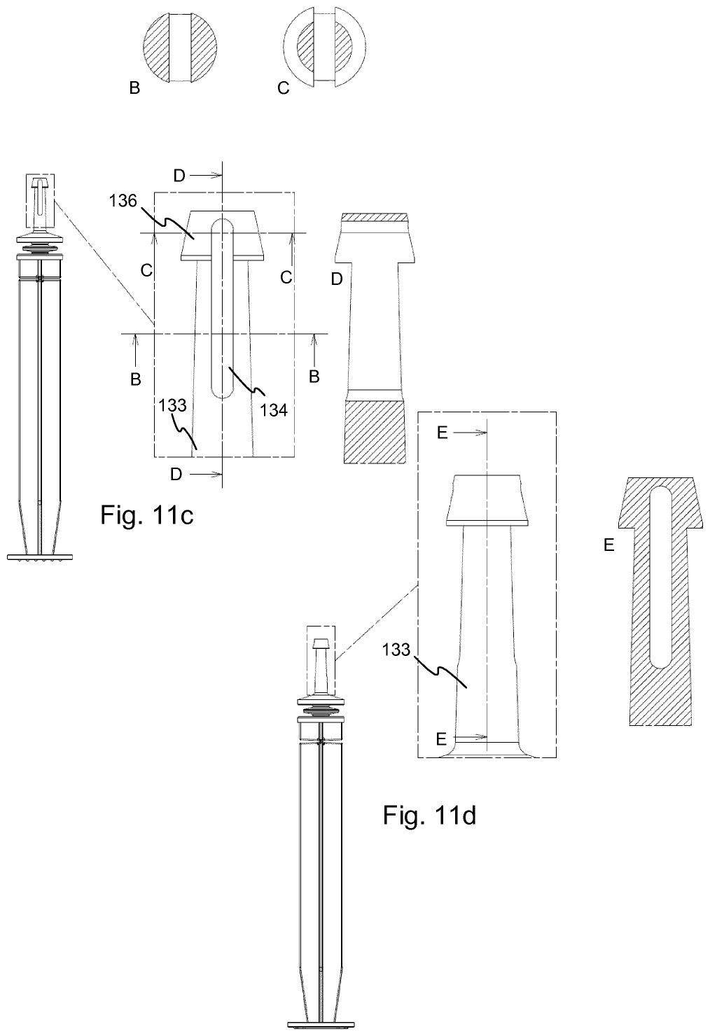

FIGS. 11a-d show views of the protrusion 133 according to one example, where the elongated recess 134 of the protrusion 133 does not have rounded edges.

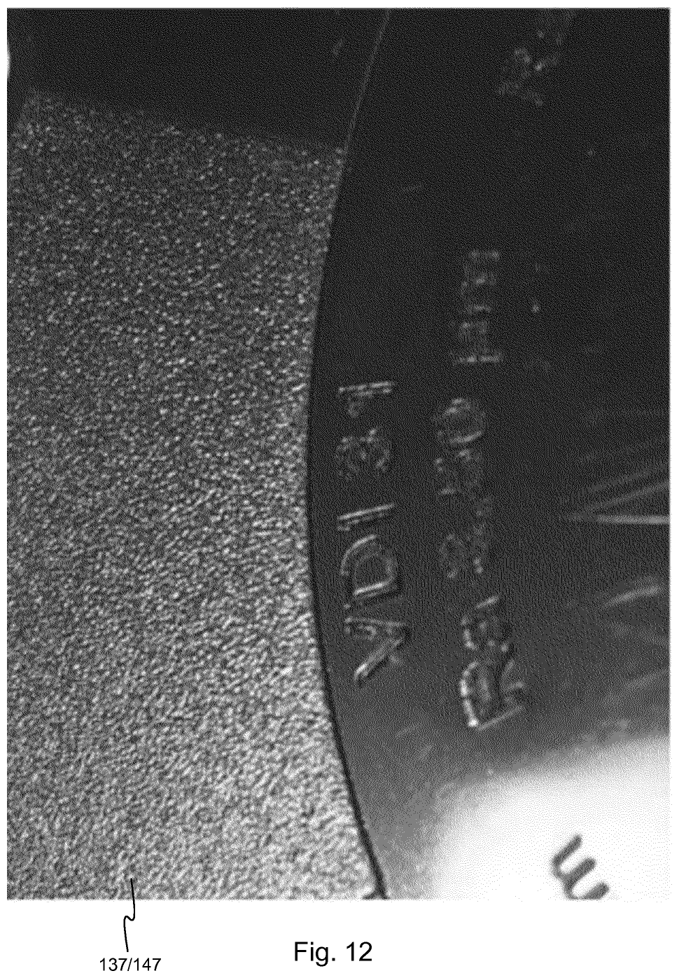

FIG. 12 shows a distal view of the proximally facing interaction surface 137 of the retaining portion 136 according to one example, where it has a surface roughness arithmetic average (Ra) of 3.5 μm. One or both of the interaction surfaces 137, 147 may have an increased surface roughness. Preferably, at least the proximally facing interaction surface 147 has an increased surface roughness. This is used to add friction force between the interaction surfaces 137, 147 to prevent them from sliding apart from each other when the plunger 196 is pulled backwards, i.e. in a proximal direction.

The increased surface roughness may have a Ra between 1.1-9 μm. The Ra must be above 1.1 μm as otherwise the surface/s is/are not rough enough to prevent sliding. The Ra must be below 9 μm as the interaction surfaces 137, 147 are not large enough to accommodate a larger Ra.

The entire area of the interaction surfaces 137, 147 do not need to have an increased surface roughness. However, a bigger area leads to a greater retaining effect for the interaction surfaces 137, 147. By increasing the surface roughness, smaller areas of the interaction surfaces 137, 147 are needed to achieve a sufficient retaining effect, which also means that the wall thickness of the surrounding areas may be reduced. This makes the syringe 100 faster and cheaper to manufacture using e.g. injection molding. Both of the interaction surfaces 137, 147 do not need to have the same Ra.

The increased surface roughness is preferably achieved using sparking. Sparking is cheapest, yields the least wear and gives the best results. Alternatives include etching or blasting.

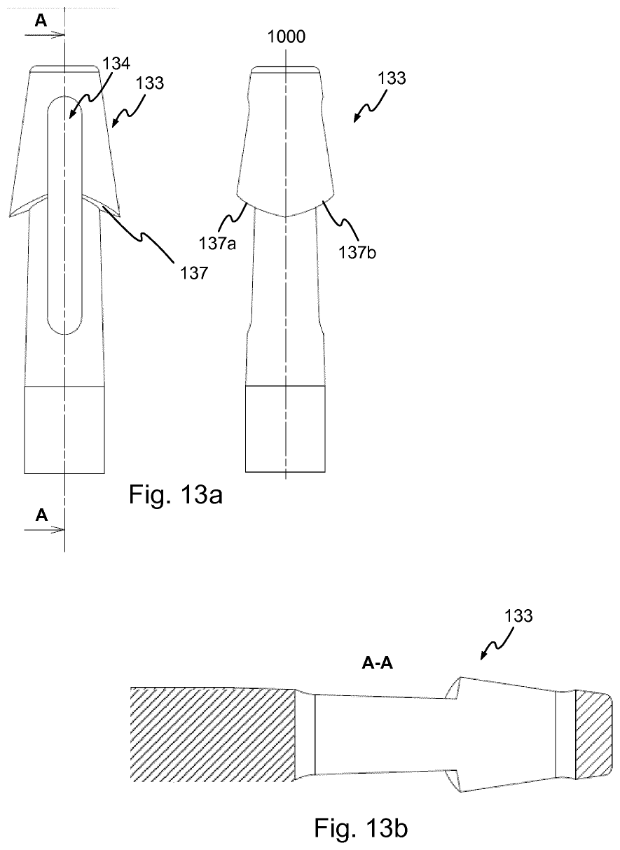

FIGS. 13a-b are views of the protrusion 133 according to one example. FIG. 13b shows a longitudinal section view along A-A of FIG. 13a. In this and most examples, the protrusion 133 comprises two proximally facing interaction surfaces 137, each extending outwards in opposite directions from an elongated recess 134 that penetrates the protrusion 133 perpendicularly to the extending directions of the interaction surfaces 137. In this example, each proximally facing interaction surface 137 of the retaining portion 136 of the protrusion 133 is divided into a first 137a and second 137b interaction surface. The first 137a and second 137b interaction surfaces have different angles towards the longitudinal axis 1000 of the protrusion 133. In this way, the protrusion 133 may be injection molded while simultaneously being easy to eject from the injection mold cavity.

Also, this is beneficial in that it is very important to prevent outside manipulation of the interaction surfaces 137, 147. By giving the first 137a and second 137b interaction surfaces different angles towards the protrusion 133, it is much harder to reduce the retaining effect of the interaction surfaces 137, 147 e.g. by poking a stick into the syringe head 140 and pushing the protrusion 133 transversally to its longitudinal axis 1000.

Further, deformation due to proximal force cannot be allowed to reduce the retaining effect of the interaction surfaces 137, 147. By e.g. angling the first 137a and second 137b interaction surfaces towards each other, this and other deformations are reduced by making use of the stiffness of the protrusion 133.

An additional benefit is that the first 137a and second 137b interaction surfaces are simpler to manufacture if they have different angles towards the protrusion. This allows for finer details, a harder and/or stiffer material to be used, which in turn further improves the retaining effect of the interaction surfaces 137, 147.

In one example, the protrusion 133 is made from a softer material, such as polypropylene with an E-modulus of 800-1200 MPa, and the distally facing interaction surface 147 of the retaining wall 148 is made from a harder material, such as polypropylene with an E-modulus of 1500-2000 MPa. If the protrusion then comprises first 137a and second 137b interaction surfaces angled towards each other while the distally facing interaction surface 147 does not comprise differently angled surfaces, a proximal force will deform the first 137a and second 137b interaction surfaces to the shape of the distally facing interaction surface 147. This will increase the area of contact between the interaction surfaces 137, 147, further improving the retaining effect.

The first 137a and second 137b interaction surfaces may additionally or alternatively have different distances to the distal tip of the protrusion 133. This is beneficial in that several planes of the retaining effect of the interaction surfaces 137, 147 are usable. This may be more difficult to manipulate, as both planes may be difficult to reach at once.

Instead or in addition to a first 137a and second 137b distinct interaction surface, each interaction surface 137 may be curved. This curve may be concave, convex, or any other shape. Each interaction surface 137 may be curved differently and a single interaction surface 137 may have different curvature in different areas.

FIG. 14 shows an example where each proximally facing interaction surface 137 of the retaining portion 136 of the protrusion 133 is made in one plane. This plane extends from the elongated recess 134 in a 90° angle to the longitudinal axis 1000 along the wall of the recess 134 and outwards perpendicularly from the wall of the recess 134 in a 70° angle to the longitudinal axis 1000. A straight plane is simple to manufacture and will deform less than curved planes when being removed from e.g. a mold.

The straight plane interaction surface 137 is advantageous in that both during manufacturing and during use, deformation of the retaining portion 136 of the protrusion 133 will increase further away from the center of the protrusion 133. As the straight interaction surface 137 naturally changes the angle to the longitudinal axis 1000 as the protrusion 133 is rotated, this uneven deformation may be anticipated and the parts of the retaining portion 136 further away from the center of the protrusion 133 may be made more resilient to deformation. This is accomplished by having a lower angle towards the longitudinal axis 1000 of the protrusion 133 and a farther distance towards the distal tip of the protrusion 133 in a perpendicular direction from the elongated recess 134 of the protrusion 133 compared to other directions.

The angle between each proximally facing interaction surface 137 and a longitudinal axis 1000 of the protrusion 133 is different along their radial extension around the longitudinal axis 1000. In a first longitudinal section, along a first longitudinal section of the protrusion 133, at the point where each interaction surface 137 ends at the recess 134 going transversally through the protrusion 133, the angle between the interaction surface 137 is perpendicular to the longitudinal axis 1000 of the protrusion 133, while the same angle continuously decreases radially along the interaction surface 137 to a point on the interaction surface 137, along a second longitudinal section of the protrusion 133 being perpendicular to the first longitudinal section. In FIG. 14, this angle decreases from 90° to 70° during this rotation, however different intervals are possible such as from 90° to 60° or 80° to 70°. In this way, the protrusion 133 may be injection molded while simultaneously being easy to eject from the injection mold cavity.

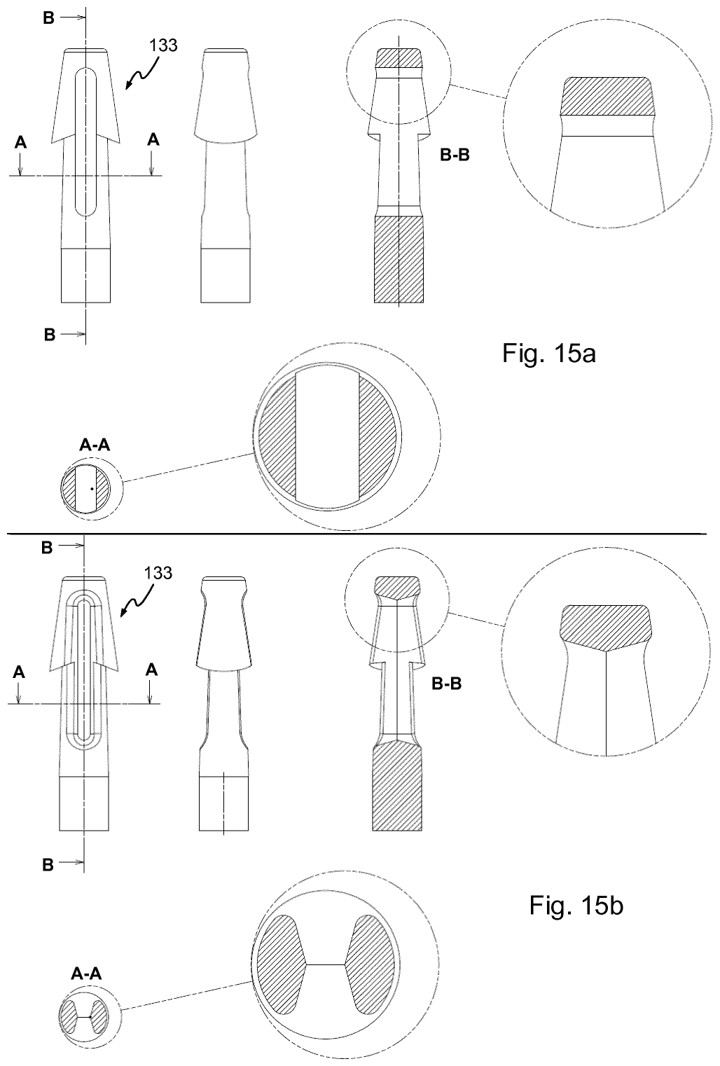

FIGS. 15a-b show the protrusion 133 according to different examples. In FIG. 15a, the elongated recess 134 of the protrusion 133 does not have rounded edges and in FIG. 15b, the elongated recess 134 has rounded edges.

The elongated recess 134 as discussed previously allows for an elastic compression of the protrusion 133 so that it may be pressed beyond the retaining wall 148 of the syringe 100 in order to administer the medicament contained within the syringe 100. Once the proximally facing interaction surface 137 has reached beyond this point, the compression allowed by the elongated recess 134 will yield a spring force that returns the interaction surface 137 to its interlocking position.

The spring force is affected by the existence of rounded edges. The angles of the rounding will also affect the thickness of the walls of the protrusion 133 around the recess 134. The rounded edges decrease the thickness of the walls of the protrusion 133, which in turn increase the flexibility of the protrusion 133, which allows for greater compression. Further, the strength and direction of the spring force will depend on the angles of the rounded edges of the elongated recess 134, which are seen e.g. in cross-section A-A of FIG. 15b.

By providing the interlocking mechanism a distance away from the distal end of the syringe 100, it is much harder to avoid triggering the interlocking mechanism by simply not extending the plunger 196 to the position where the interaction surface 137 returns to its interlocking position. For one, it may be unexpected that the interlocking mechanism occurs before all medicament is administered. Furthermore, by designing the elongated recess 134 and the shape of the protrusion 133 such that the elastic compression occurs smoothly without alerting the user, it may be more difficult to abuse. Additionally, the visibility of the interaction surfaces 137 may be obscured from view.

The most important way to dissuade the user to avoid triggering the interlocking mechanism is however, that by providing it a distance away from the distal end of the syringe 100, expensive medicament is wasted if it is not triggered.

The downside of this added distance is that more volume of the syringe 100 is wasted that could have otherwise contained more medicament. However, a reusable syringe will never use all of its volume for medicament as air needs to be removed after manual filling of medicament. The inventor has therefore deemed this downside to be negligible.

Air needs to be removed from the syringe 100 because injecting air bubbles may be very dangerous. The rounded edges of the elongated recess 134 of FIG. 15b further wastes volume of the syringe 100, however they are advantageous in that it is easier for the user to remove any air bubbles. The angles of the rounded edges and the reduced wall thickness of the protrusion 133 alleviates the removal of air bubbles. This is because sharp edges allow for air bubbles to be trapped.

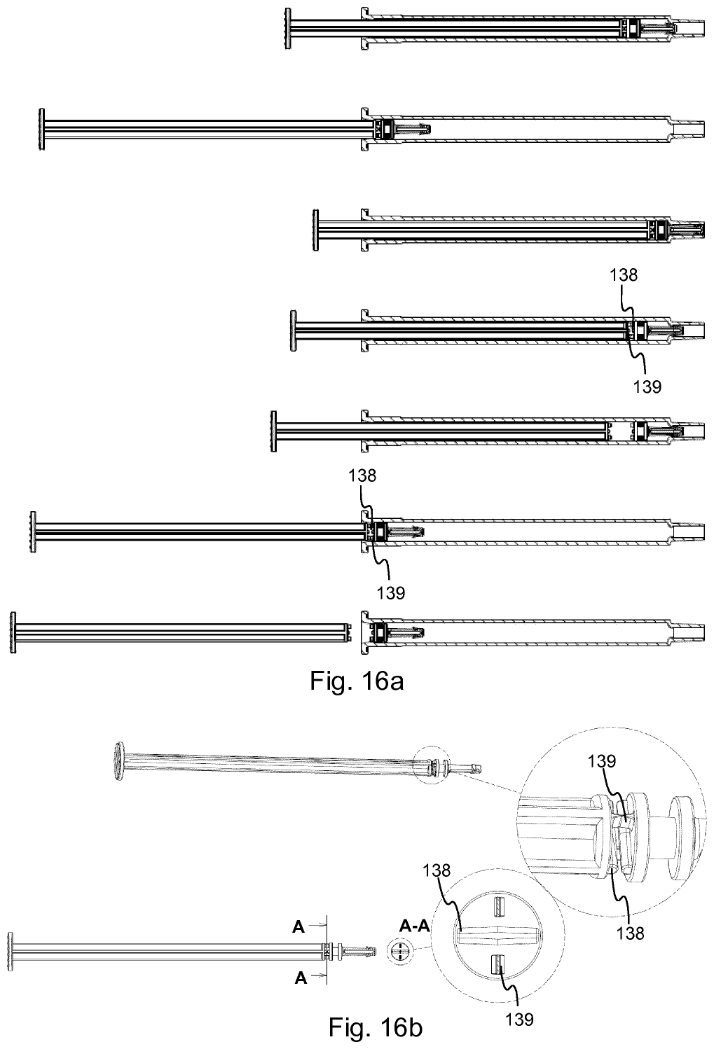

FIGS. 16a-b and 17a-b show different examples of an embodiment where the plunger 196 comprises two kerfs 139 in a cavity of the plunger 196 and ridges 138 arranged to support the cavity.

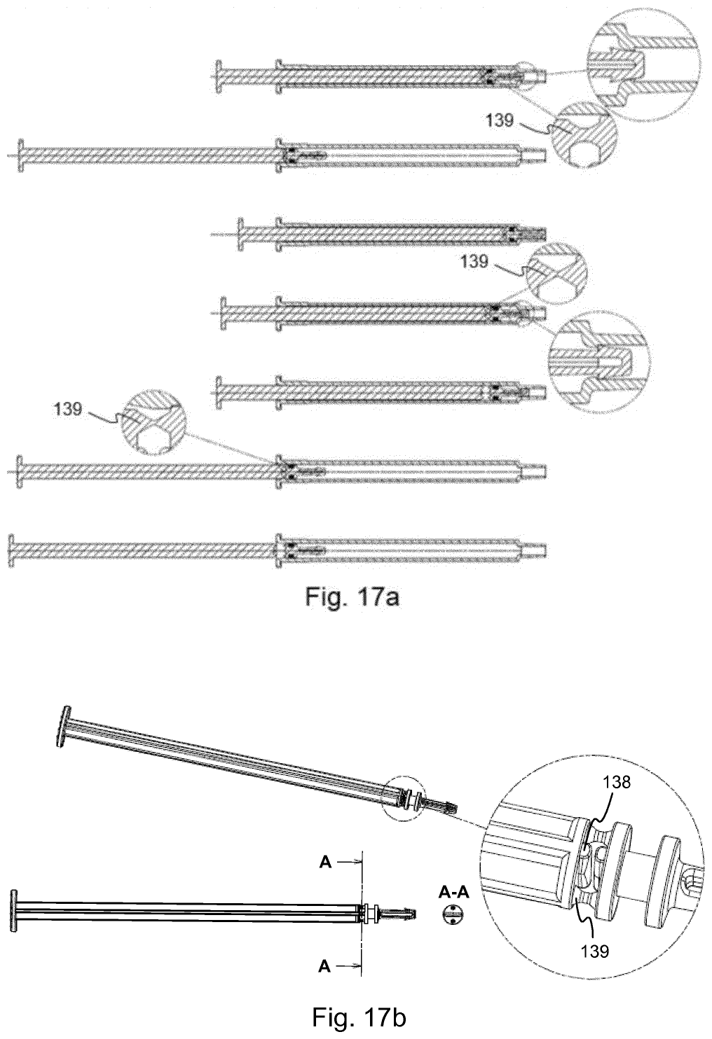

The kerfs 139 in FIG. 16a-b comprise two joined wedges with a thin middle section arranged to release the two wedges from each under a suitable strength proximal force. The kerfs 139 in FIGS. 17a-b comprise two joined, truncated cones with a thin middle section arranged to induce necking and eventually breaking under a suitable strength proximal force. FIG. 17a shows different stages of necking and finally a broken kerf 139.

As a further example not shown, the kerfs 139 may comprise two hard plastic discs joined by an adhesive arranged to release the two discs from each under a suitable strength proximal force.

The kerfs 139 of these examples are arranged near two different edges of a cross-section of the plunger 196, as is shown in cross-section A-A of FIGS. 16-17b. Two kerfs 139 are advantageous over one in that it makes the plunger 196 more stable during manufacturing, transportation and use. As the kerfs 139 by design are made to break, they are structurally weaker than the rest of the syringe 100.

By having more than one kerf 139, it not only increases the total strength of the plunger 196, but also reduces the amount of axes from which the structure is weak to pressure. Looking to the cross-section A-A of FIG. 17b as an example, the kerfs 139 are more resilient to pressure in a vertical direction than a horizontal direction.

Importantly, having more than one kerf 139 does not negatively influence the production speed. Several kerfs 139, such as two, three or four, may be produced at once and yield a robust plunger 196. The resulting syringe 100 does not have a production that is more complex or includes more cavities than a standard three-component syringe, and may therefore be produced as quickly and cheaply as reusable syringes. Hence, a competitive non-reusable alternative to reusable syringes is possible.

In order to further improve the stability of the plunger 196, ridges 138 may be used. Ridges 138 may be any shape such as oblong or circular protrusions and made from any material, however preferably a hard material at least harder than the kerf(s) 139.

In the cross-section A-A of FIG. 17b, ridges 138 are used to make the plunger 196 more resilient in the otherwise weak horizontal direction as discussed previously.

The shown examples comprise two ridges 138 arranged near the kerf(s) 139. While all examples shown use two ridges 138, any number may be used such as one or three. These ridges 138 will interact under pressure in order to prevent an unintended break of the plunger 196 e.g. during manufacturing. Ridges 138 may be likened to pre-broken kerfs 139, in that they do not modify the proximal force needed to intentionally break the plunger 196, but they prevent bending perpendicular to this force.

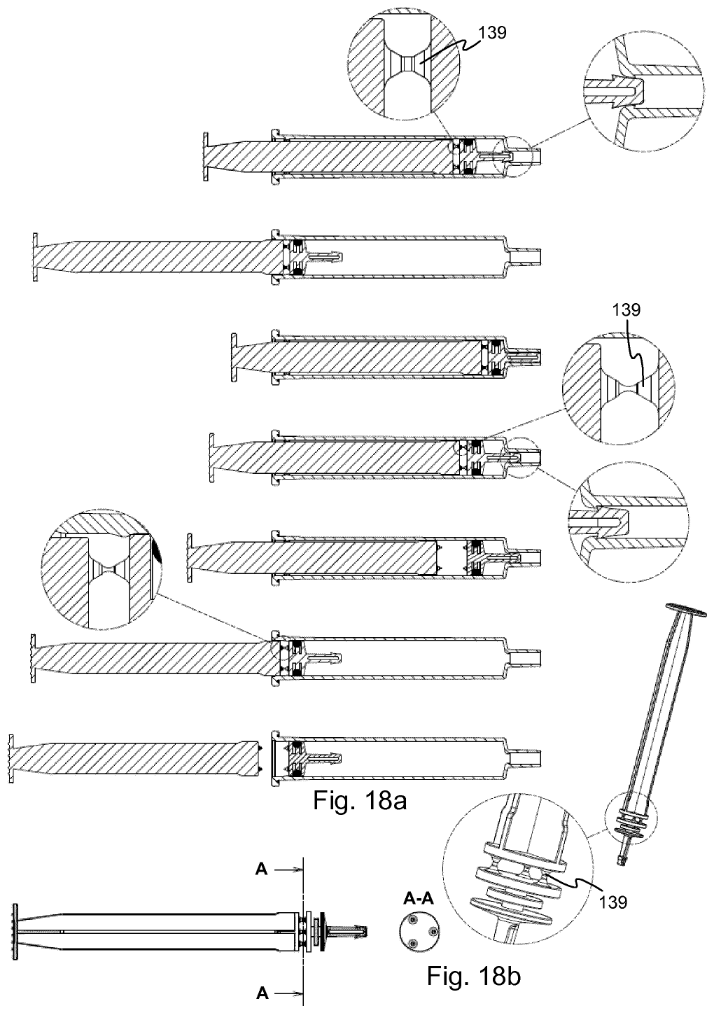

FIGS. 18a-b show an example syringe 100 during different stages of use, where the plunger 196 comprises three kerfs 139. The kerfs 139 are arranged near three different edges of a cross-section of the plunger 196. They are arranged 120° from each other. It is beneficial to arrange the kerfs 139 symmetrically in order to load the applied force equally to each of the kerfs 139.

FIG. 18b further shows at least one disc arranged at the distal end of the plunger 196 with a diameter at least large enough to disallow proximal removal of the plunger 196 from the barrel 111 of the syringe 100. When proximal removal is attempted, at least one disc is caught on the barrel 111 e.g. on a protrusion as shown in FIG. 18a. The plunger 196 is configured to break if further proximal force is applied, as shown in FIG. 18a.

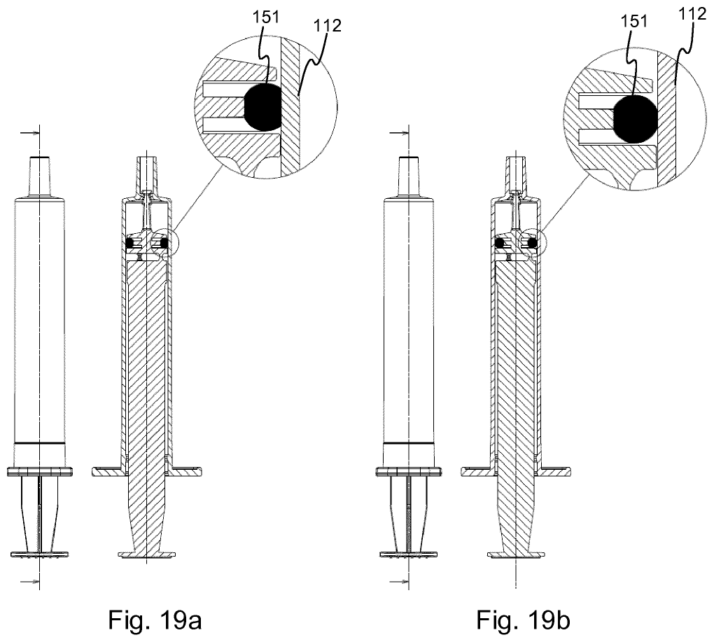

FIGS. 19a-b shows different embodiments of the syringe 100 with different O-rings 151. The O-ring 151 may be made of e.g. elastomers or synthetic rubber, but is preferably made of silicon. Silicon is beneficial in that it is more inert to the medicament than the alternatives, which ensures the purity of the medicament for a long time, in a magnitude of years.

Cylindrical steel adapters are used to shape and fit the O-ring 151 to a desired shape and position before being mounted in the Syringe 100.

The silicon preferably has a hardness of 50 to 100 IRHD, even more preferably 67 to 77 IRHD and most preferably 72 IRHD. The hardness may be measured in any suitable way, such as those set out in the standard of ISO 48. Prior art usually has a hardness of less than 50 IRHD. The high hardness degree of the silicon is beneficial in that it greatly reduces the need for silicon lubrication, which may contaminate the medicament in the syringe 100.

The O-ring 151 may be press fit radially 0.1 to 0.2 mm, preferably 0.15 mm corresponding to the embodiment of FIG. 19b. This yields a smaller contact area between the O-ring 151 and the tubular wall 112 than prior art conventions, which are press fit radially over 0.5 mm corresponding to the embodiment of FIG. 19a. The smaller contact area is beneficial in that a more consistent seal is achieved with a higher pressure difference allowed on each side of the O-ring 151 and lower friction will occur during movement along the axis 1000 of the extension of the barrel 111, e.g. during normal use.

Further, the invention has mainly been described with reference to a few embodiments. However, as is readily understood by a person skilled in the art, other embodiments than the ones disclosed above are equally possible within the scope of the invention, as defined by the appended claims.

1. A syringe comprising:
a barrel with a barrel lumen, the barrel being adapted to contain a drug, the barrel having a distal end wall portion and a tubular wall extending proximally from the distal end wall portion, the barrel extending along an axis;
wherein the distal end wall portion has an opening extending into a syringe head adapted to receive a syringe needle, the syringe head having a syringe head lumen smaller in transversal cross-section than the barrel lumen;
a plunger extending along the axis, the plunger being displaceable along the axis within the barrel;
wherein the plunger includes a plunger top, the plunger top being adapted to be in fluid-tight engagement with the tubular wall of the barrel;
wherein the plunger top includes a protrusion that is adapted to fit in the syringe head lumen;
wherein the protrusion has an elongated recess extending along the axis, and a retaining portion that is wider in transversal cross-section than the remainder of the protrusion;
wherein the protrusion is adapted to be retained inside the syringe head lumen upon insertion by a proximally facing interaction surface;
wherein the syringe head includes a retaining wall having a distally facing interaction surface, and the plunger is adapted to be prevented from being pulled in a direction away from the distal end wall portion by the proximally facing interaction surface of the retaining portion locking against the distally facing interaction surface of the retaining wall;
wherein the retaining wall forms a section of the syringe lumen that is smaller in transversal cross-section than a remainder of the syringe; and
wherein the proximally facing and the distally facing interaction surfaces are arranged to engage with each other when the plunger top is at a distance (D2) from the distal end wall portion.

2. The syringe according to claim 1, wherein the distance (D2) is between 0.1-7.5 mm.

3. The syringe according to claim 1, wherein the proximally facing and the distally facing interaction surfaces are arranged to engage with each other when a distal end of the protrusion is at a distance (D1) of between 0.1-7.5 mm from a distal end of the syringe head.

4. The syringe according to claim 3, wherein the distances (D1, D2) are equal.

5. A syringe comprising:
a barrel with a barrel lumen, the barrel being adapted to contain a drug, the barrel having a distal end wall portion and a tubular wall extending proximally from the distal end wall portion, the barrel extending along an axis;
wherein the distal end wall portion has an opening extending into a syringe head adapted to receive a syringe needle, the syringe head having a syringe head lumen smaller in transversal cross-section than the barrel lumen;
a plunger extending along the axis, the plunger being displaceable along the axis within the barrel;
wherein the plunger including a plunger top, the plunger top being adapted to be in fluid-tight engagement with the tubular wall of the barrel;
wherein the plunger top includes a protrusion that is adapted to fit in the syringe head lumen;
wherein the protrusion has an elongated recess extending along the axis, and a retaining portion that is wider in transversal cross-section than the remainder of the protrusion;
wherein the protrusion is adapted to be retained inside the syringe head lumen upon insertion by a proximally facing interaction surface;
wherein the syringe head includes a retaining wall having a distally facing interaction surface, and the plunger is adapted to be prevented from being pulled in a direction away from the distal end wall portion by the proximally facing interaction surface of the retaining portion locking against the distally facing interaction surface of the retaining wall; and
wherein at least one of the proximally facing and the distally facing interaction surfaces has a surface roughness arithmetic average (Ra) between 1.1-9 μm.

6. The syringe according to claim 5, wherein the surface roughness of the at least one of the proximally facing and the distally facing interaction surfaces is achieved using sparking, etching or blasting.

7. A syringe comprising:
a barrel with a barrel lumen, the barrel being adapted to contain a drug, the barrel having a distal end wall portion and a tubular wall extending proximally from the distal end wall portion, the barrel extending along an axis;
wherein the distal end wall portion has an opening extending into a syringe head adapted to receive a syringe needle, the syringe head having a syringe head lumen smaller in transversal cross-section than the barrel lumen;
plunger extending along the axis, the plunger being displaceable along the axis within the barrel;
wherein the plunger includes a plunger top, the plunger top being adapted to be in fluid-tight engagement with the tubular wall of the barrel;
wherein the plunger top includes a protrusion that is adapted to fit in the syringe head lumen;
wherein the protrusion has an elongated recess extending along the axis, and a retaining portion that is wider in transversal cross-section than the remainder of the protrusion;
wherein the protrusion is adapted to be retained inside the syringe head lumen upon insertion by a proximally facing interaction surface;
wherein the syringe head includes a retaining wall having a distally facing interaction surface, and the plunger is adapted to be prevented from being pulled in a direction away from the distal end wall portion by the proximally facing interaction surface of the retaining portion locking against the distally facing interaction surface of the retaining wall; and
wherein the elongated recess of the protrusion has rounded edges.

8. The syringe according to claim 7, wherein the rounded edges create divots in a radius of a top of the retaining portion

9.-18. (canceled)

19. The syringe according to claim 1, wherein at least one of the proximally facing and the distally facing interaction surfaces has a surface roughness arithmetic average (Ra) between 1.1-9 μm; and
wherein the elongated recess of the protrusion has rounded edges.

20. The syringe according to claim 1, wherein after the proximally facing and the distally facing interaction surfaces have engaged with each other, a distal end of the protrusion is displaceable along the axis between a position of interaction surface engagement and a distal end of the syringe head.

21. The syringe according to claim 1, wherein the plunger comprises at least one kerf adapted to deform or break when the plunger is pulled in a direction away from the distal end wall portion after retaining of the protrusion of the plunger top inside the swinge head lumen so as to disallow displacement of the plunger top by movement of the plunger.

22. The syringe according to claim 21, wherein the kerf is arranged along the axis at a distance (D3) away from the plunger top; and
wherein the distance (D3) is shorter than a distance (D4) between a proximal end of the barrel and a stopping portion of the tubular wall.

23. The syringe according to claim 21, wherein the plunger is made of a rigid material adapted to break at the kerf when the plunger is pulled in the direction away from the distal end wall portion.

24. The syringe according to claim 23, wherein the plunger is made of Polypropylene with a relatively high E-modulus.

25. The syringe according to claim 21, wherein the plunger is made of an elastic material adapted to viscoelastically deform at the kerf when the plunger is pulled in the direction away from the distal end wall portion.

26. The syringe according to claim 25, wherein the plunger is made of Polypropylene with a relatively low E-modulus.

27. The syringe according to claim 1, wherein the distally facing interaction surface of the retaining wall and/or the proximally facing interaction surface of the retaining portion extend in an inclined direction extending proximally from the axis.

28. The syringe according to claim 1, wherein the proximally facing interaction surface of the retaining portion of the protrusion and the distally facing interaction surface of the retaining wall are disposed so as to allow for proximal displacement of the plunger before retaining contact between the proximally facing interaction surface of the retaining portion and the distally facing interaction surface of the retaining wall occurs.

29. The syringe according to claim 1, wherein the distal end wall portion of the barrel comprises reinforcing ribs.

30. The syringe according to claim 1, wherein the elongated recess is substantially conical and wider in transversal cross-section towards a distal tip of the protrusion.

31.-37. (canceled)
